(19) United States
(12) Patent Application Publication
(10) Pub. No.: US 2025/0002529 A1
(43) Pub. Date: Jan. 2, 2025

(54) NOVEL DEUTERATED CYANO COMPOUNDS, PREPARATION METHODS, COMPOSITIONS AND APPLICATIONS

(71) Applicant: Guangzhou Anobri Pharmaceutical Co. Ltd., Guangdong (CN)

(72) Inventors: Cai Gu Huang, Guangdong (CN); Yong Hua Deng, Guangdong (CN); Hui Sun, Guangdong (CN)

(21) Appl. No.: 18/699,969

(22) PCT Filed: Apr. 1, 2022
(86) PCT No.: PCT/CN2022/084709

§ 371 Date: Apr. 10, 2024

(30) Foreign Application Priority Data
Oct. 22, 2021 (CN) 202111234708.X

(51) Int. Cl. C07K 5/062; A61K 38/00; A61P 31/14

(52) U.S. Cl. CPC C07K 5/06017; A61P 31/14; C07K 5/06034; A61K 38/00

(57) ABSTRACT

Deuterated cyano compounds useful as a 3CL protease inhibitor, methods of making the compounds, pharmaceutical compositions containing the compounds, and methods of treating a viral infection in a human by administering the compounds.

### CROSS-REFERENCE TO RELATED APPLICATIONS

This application is a national stage filling under 35 U.S.C. § 371 of international application number PCT/CN2022/084709, filed Apr. 1, 2022, which claims priority to Chinese patent application No. 202111234708.X, filed Oct. 22, 2021. The contents of these applications are incorporated herein by reference in their entirety.

### BACKGROUND OF THE INVENTION

The coronavirus in humans was first isolated in the United Kingdom in the 1960s and was named after a crown-shaped protrusion on its surface. It may be associated with respiratory infections in humans, pigs, cats, dogs, mice, and chickens.

SARS virus belongs to the order of nested viruses, Coronaviridae and Coronaviruses, and is a subset B Coronavirus of β genus. The virions are enveloped, and surrounded by coronal filaments, distributed in the cytoplasm and are round in shape. The diameter of the virus ranges from 80 nm to 120 nm. SARS is an infectious disease with rapid onset, rapid spread and high mortality. Most patients are infected by direct or indirect contact with an already infected patient, or live in endemic areas. MERS-CoV is a subset C Coronavirus of β genus, which is named Middle East Respiratory Syndrome Coronavirus (MERS-CoV). MERS-CoV was first discovered in Saudi Arabia in September 2012. MERS-CoV was named “SARS-like virus” because of its clinical symptoms similar to SARS, and became the sixth known human Coronavirus and the third species isolated in the past 10 years. The coronavirus SARS-CoV-2 is a novel coronavirus strain that has never been previously found in humans. It was first discovered and reported in 2019. It is still prevalent over the world and is not well controlled in regions of many countries.

The common signs of coronavirus infection include respiratory symptoms, fever, cough, shortness of breath, and dyspnea. In more severe cases, infection can lead to pneumonia, severe acute respiratory syndrome, renal failure, and even death, and currently there is no specific treatment for diseases caused by coronaviruses.

In 2021, Dafydd R. Owen published the paper “An Oral SARS-CoV-2 Mpro Inhibitor Clinical Candidate for the Treatment of COVID-19”, describing the pharmacodynamic effect of the 3CL protease inhibitor-PF-07321332 as a viral inhibitor. PF-07321332 prevents the virus from cleaving a long protein chain that is required for its self-replication into parts by inhibiting the main protease. The compound is being evaluated for clinical efficacy in the treatment of COVID-19 in Phase 3 clinical trials. Generally, this polypeptide compound PF-07321332 needs to be combined with other drugs (protease protectants) to improve its pharmacokinetic properties in vivo due to its easy metabolic decomposition by first-pass metabolism. Thus the pharmacokinetic and metabolic properties of PF-07321332 need to be improved.

Therefore, there is a need in the art to develop novel 3CL protease inhibitor compounds with better inhibitory activity or pharmacokinetic properties. The present invention designs and discloses novel deuterated cyano compounds, which achieve better pharmacokinetic properties and therapeutic effects, and better drug-forming properties, than PF-07321332 on the basis of comparable virologic inhibitor activities. Methods of preparing the compounds, pharmaceutical compositions containing the compounds, and applications using the compounds are disclosed. Scaled up production of the compounds and drugs containing the compounds can be realized. The compounds have good clinical value.

### SUMMARY OF THE INVENTION

The invention belongs to the medical field, in particular to novel deuterated cyano compounds, their preparation, compositions containing the compounds, and applications using the compounds. The compounds are used to prepare pharmaceutical compositions for treatment and prevention of viral infections.

### DETAILED DESCRIPTION OF THE INVENTION

The invention provides novel deuterated cyano compounds, preparation methods, compositions containing the compounds, and applications using the compounds.

The invention is directed to deuterated cyano compounds represented by Formula I, or a pharmaceutically acceptable salt, isomer, or prodrug thereof:

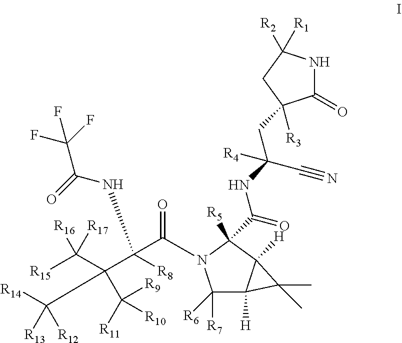

wherein R₁ to R₁₇ are a combination of hydrogen isotopes (including the isotopes “protium” and “deuterium”); and
wherein at least one of R₁ to R₁₇ is deuterium, and pharmaceutically acceptable salts, isomers, and prodrugs thereof.

In one embodiment, at least one of R₁ to R₂ is deuterium in the compound of formula I.

In one embodiment, at least one of R₄ to R₅ is deuterium in the compound of formula I.

In one embodiment, at least one of R₆ to R₇ is deuterium in the compound of formula I.

In one embodiment, R₈ is deuterium in the compound of formula I.

In one embodiment, each of R₉ to R₁₇ are deuterium in the compound of formula I.

In one embodiment, at least one of R₁ to R₂ is deuterium, at least one of R₆ to R₇ is deuterium, and R₉ to R₁₇ are all deuterium in the compound of formula I.

The invention is also directed to a method for preparing the deuterated cyano compounds comprising the following steps:

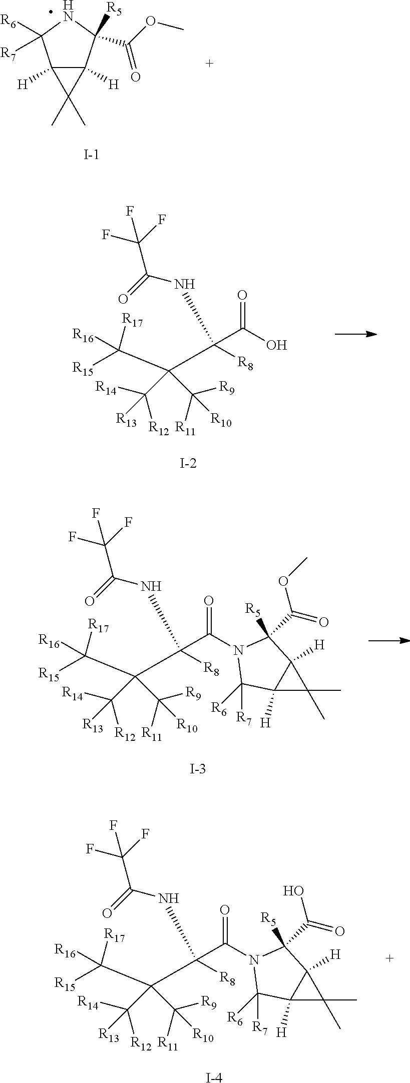

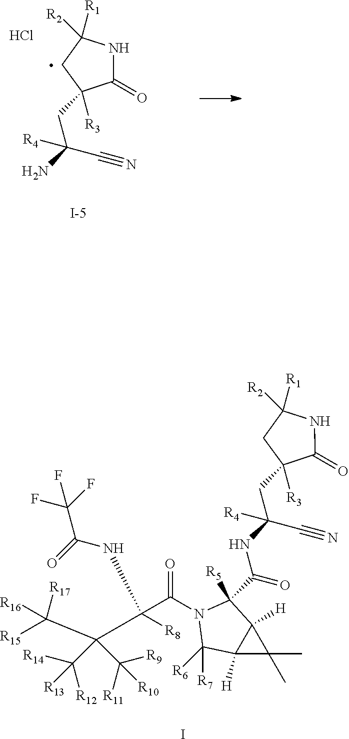

(1) intermediate I-3 is obtained by condensing compound I-1 with compound I-2 by the action of a condensing agent in an organic solvent;
(2) intermediate I-4 is obtained by the hydrolyzing intermediate I-3 under the action of alkali in an organic solvent; and
(3), compound I is obtained by condensing compound I-4 with compound I-5 by the action of condensing agent in an organic solvent.

In certain embodiments, the invention includes pharmaceutical compositions characterized in that they contain a pharmaceutically acceptable carrier and a compound of formula I, or a pharmaceutically acceptable salt thereof.

In certain embodiments, the invention is directed to a pharmaceutical composition characterized in that it includes an additional therapeutic agent which is an antiviral drug.

The invention is directed to the use of the above-mentioned pharmaceutical compositions to prepare a 3CL protease inhibitor.

The invention is directed to the use of the pharmaceutical compositions to treat a viral infection in humans, wherein the virus is selected from the group consisting of human coronavirus, novel coronavirus (SARS-CoV-2), SARS coronavirus, and MERS coronavirus.

In various embodiments, the compound has one of the structures shown below in Table 1.

TABLE 1
Representative Compounds

| Sr. | |
|---|---|
| No. | Structure |
| 1 | 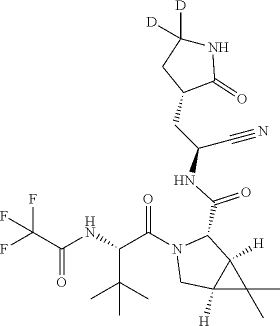 [SMILES: [2H]C1([2H])C[C@@H](C[C@@H](C#N)NC(=O)[C@@H]2[C@@H]3[C@H](CN2C(=O)[C@@H](NC(=O)C(F)(F)F)C(C)(C)C)C3(C)C)C(=O)N1] |
| 2 | 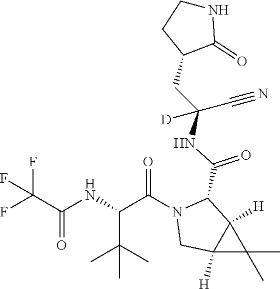 [SMILES: [2H][C@@](C#N)(C[C@@H]1CCNC1=O)NC(=O)[C@@H]1[C@@H]2[C@H](CN1C(=O)[C@@H](NC(=O)C(F)(F)F)C(C)(C)C)C2(C)C] |
| 3 | 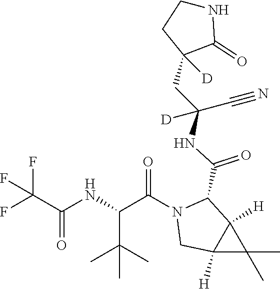 [SMILES: [2H][C@@]1(C[C@@]([2H])(C#N)NC(=O)[C@@H]2[C@@H]3[C@H](CN2C(=O)[C@@H](NC(=O)C(F)(F)F)C(C)(C)C)C3(C)C)CCNC1=O] |
| 4 | 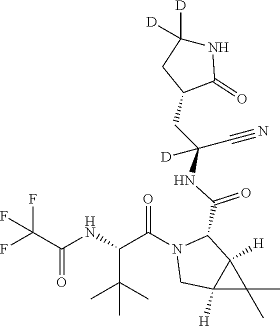 [SMILES: [2H]C1([2H])C[C@@H](C[C@@]([2H])(C#N)NC(=O)[C@@H]2[C@@H]3[C@H](CN2C(=O)[C@@H](NC(=O)C(F)(F)F)C(C)(C)C)C3(C)C)C(=O)N1] |
| 5 | 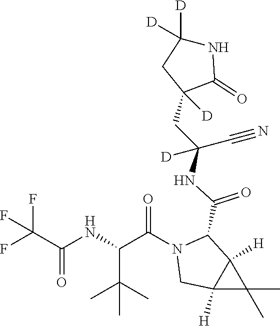 [SMILES: [2H]C1([2H])C[C@@]([2H])(C[C@@]([2H])(C#N)NC(=O)[C@@H]2[C@@H]3[C@H](CN2C(=O)[C@@H](NC(=O)C(F)(F)F)C(C)(C)C)C3(C)C)C(=O)N1] |
| 6 | 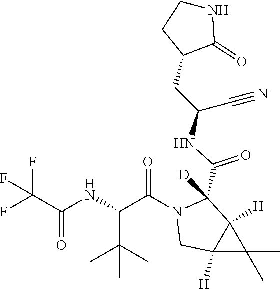 [SMILES: [2H][C@@]1(C(=O)N[C@H](C#N)C[C@@H]2CCNC2=O)[C@@H]2[C@H](CN1C(=O)[C@@H](NC(=O)C(F)(F)F)C(C)(C)C)C2(C)C] |
| 7 | 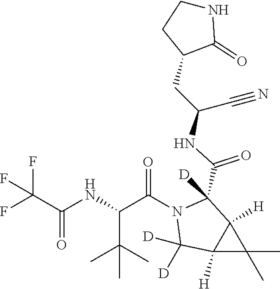 [SMILES: [2H]C1([2H])[C@H]2[C@H](C2(C)C)[C@@]([2H])(C(=O)N[C@H](C#N)C[C@@H]2CCNC2=O)N1C(=O)[C@@H](NC(=O)C(F)(F)F)C(C)(C)C] |
| 8 | 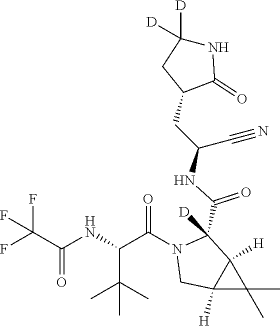 [SMILES: [2H]C1([2H])C[C@@H](C[C@@H](C#N)NC(=O)[C@]2([2H])[C@@H]3[C@H](CN2C(=O)[C@@H](NC(=O)C(F)(F)F)C(C)(C)C)C3(C)C)C(=O)N1] |
| 9 | 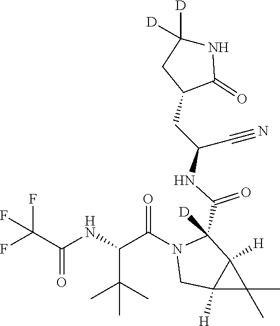 [SMILES: [2H]C1([2H])C[C@@H](C[C@@H](C#N)NC(=O)[C@]2([2H])[C@@H]3[C@H](CN2C(=O)[C@@H](NC(=O)C(F)(F)F)C(C)(C)C)C3(C)C)C(=O)N1] |
| 10 | 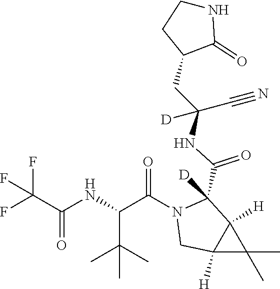 [SMILES: [2H][C@@](C#N)(C[C@@H]1CCNC1=O)NC(=O)[C@]1([2H])[C@@H]2[C@H](CN1C(=O)[C@@H](NC(=O)C(F)(F)F)C(C)(C)C)C2(C)C] |
| 11 | 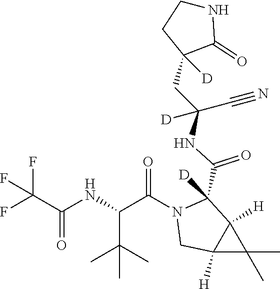 [SMILES: [2H][C@@]1(C[C@@]([2H])(C#N)NC(=O)[C@]2([2H])[C@@H]3[C@H](CN2C(=O)[C@@H](NC(=O)C(F)(F)F)C(C)(C)C)C3(C)C)CCNC1=O] |
| 12 | 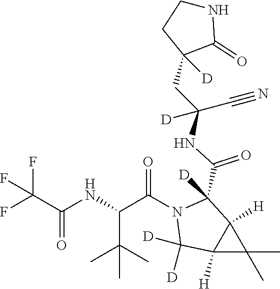 [SMILES: [2H]C1([2H])[C@H]2[C@H](C2(C)C)[C@@]([2H])(C(=O)N[C@]([2H])(C#N)C[C@]2([2H])CCNC2=O)N1C(=O)[C@@H](NC(=O)C(F)(F)F)C(C)(C)C] |
| 13 | 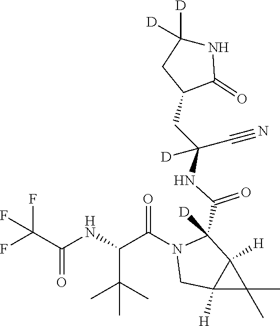 [SMILES: [2H]C1([2H])C[C@@H](C[C@@]([2H])(C#N)NC(=O)[C@]2([2H])[C@@H]3[C@H](CN2C(=O)[C@@H](NC(=O)C(F)(F)F)C(C)(C)C)C3(C)C)C(=O)N1] |
| 14 | 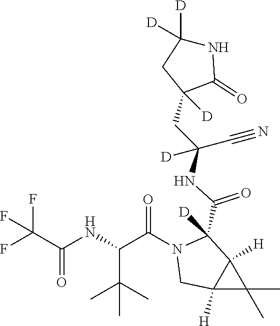 [SMILES: [2H]C1([2H])C[C@@]([2H])(C[C@@]([2H])(C#N)NC(=O)[C@]2([2H])[C@@H]3[C@H](CN2C(=O)[C@@H](NC(=O)C(F)(F)F)C(C)(C)C)C3(C)C)C(=O)N1] |
| 15 | 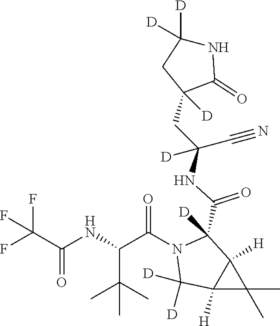 [SMILES: [2H]C1([2H])C[C@@]([2H])(C[C@@]([2H])(C#N)NC(=O)[C@]2([2H])[C@@H]3[C@@H](C3(C)C)C([2H])([2H])N2C(=O)[C@@H](NC(=O)C(F)(F)F)C(C)(C)C)C(=O)N1] |
| 16 | 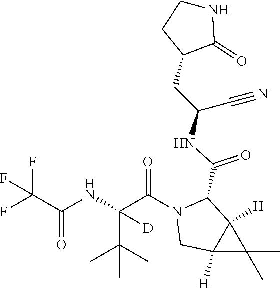 [SMILES: [2H][C@](NC(=O)C(F)(F)F)(C(=O)N1C[C@H]2[C@@H]([C@H]1C(=O)N[C@H](C#N)C[C@@H]1CCNC1=O)C2(C)C)C(C)(C)C] |
| 17 | 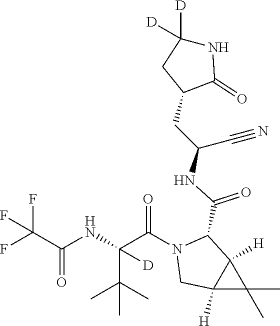 [SMILES: [2H]C1([2H])C[C@@H](C[C@@H](C#N)NC(=O)[C@@H]2[C@@H]3[C@H](CN2C(=O)[C@]([2H])(NC(=O)C(F)(F)F)C(C)(C)C)C3(C)C)C(=O)N1] |
| 18 | 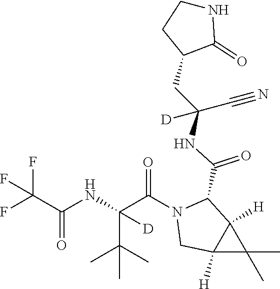 [SMILES: [2H][C@](NC(=O)C(F)(F)F)(C(=O)N1C[C@H]2[C@@H]([C@H]1C(=O)N[C@]([2H])(C#N)C[C@@H]1CCNC1=O)C2(C)C)C(C)(C)C] |
| 19 | 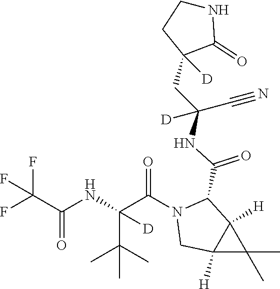 [SMILES: [2H][C@@]1(C[C@@]([2H])(C#N)NC(=O)[C@@H]2[C@@H]3[C@H](CN2C(=O)[C@]([2H])(NC(=O)C(F)(F)F)C(C)(C)C)C3(C)C)CCNC1=O] |
| 20 | 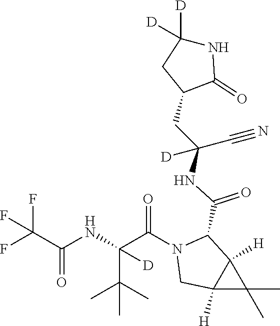 [SMILES: [2H]C1([2H])C[C@@H](C[C@@]([2H])(C#N)NC(=O)[C@@H]2[C@@H]3[C@H](CN2C(=O)[C@]([2H])(NC(=O)C(F)(F)F)C(C)(C)C)C3(C)C)C(=O)N1] |
| 21 | 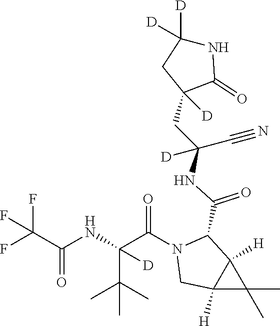 [SMILES: [2H]C1([2H])C[C@@]([2H])(C[C@@]([2H])(C#N)NC(=O)[C@@H]2[C@@H]3[C@H](CN2C(=O)[C@]([2H])(NC(=O)C(F)(F)F)C(C)(C)C)C3(C)C)C(=O)N1] |
| 22 | 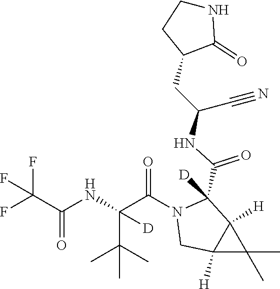 [SMILES: [2H][C@](NC(=O)C(F)(F)F)(C(=O)N1C[C@H]2[C@H](C2(C)C)[C@@]1([2H])C(=O)N[C@H](C#N)C[C@@H]1CCNC1=O)C(C)(C)C] |
| 23 | 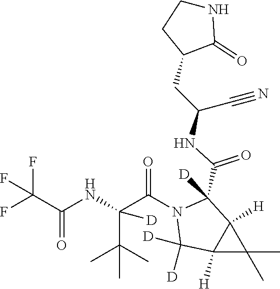 [SMILES: [2H]C1([2H])[C@H]2[C@H](C2(C)C)[C@@]([2H])(C(=O)N[C@H](C#N)C[C@@H]2CCNC2=O)N1C(=O)[C@]([2H])(NC(=O)C(F)(F)F)C(C)(C)C] |
| 24 | 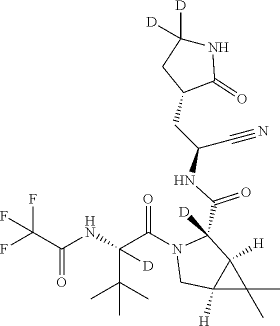 [SMILES: [2H]C1([2H])C[C@@H](C[C@@H](C#N)NC(=O)[C@]2([2H])[C@@H]3[C@H](CN2C(=O)[C@]([2H])(NC(=O)C(F)(F)F)C(C)(C)C)C3(C)C)C(=O)N1] |
| 25 | 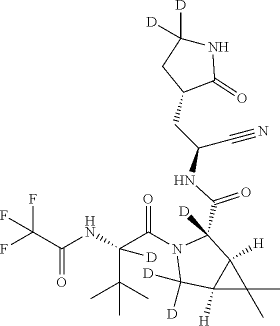 [SMILES: [2H]C1([2H])C[C@@H](C[C@@H](C#N)NC(=O)[C@]2([2H])[C@@H]3[C@@H](C3(C)C)C([2H])([2H])N2C(=O)[C@]([2H])(NC(=O)C(F)(F)F)C(C)(C)C)C(=O)N1] |
| 26 | 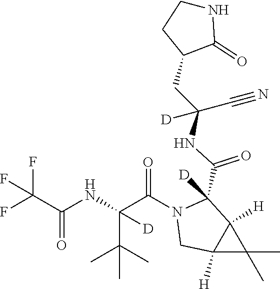 [SMILES: [2H][C@](NC(=O)C(F)(F)F)(C(=O)N1C[C@H]2[C@H](C2(C)C)[C@@]1([2H])C(=O)N[C@]([2H])(C#N)C[C@@H]1CCNC1=O)C(C)(C)C] |
| 27 | 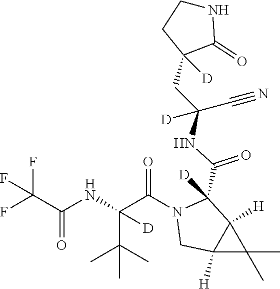 [SMILES: [2H][C@@]1(C[C@@]([2H])(C#N)NC(=O)[C@]2([2H])[C@@H]3[C@H](CN2C(=O)[C@]([2H])(NC(=O)C(F)(F)F)C(C)(C)C)C3(C)C)CCNC1=O] |
| 28 | 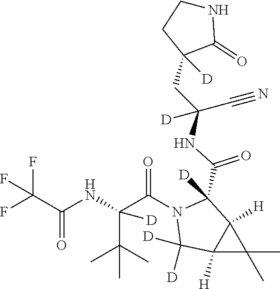 [SMILES: [2H]C1([2H])[C@H]2[C@H](C2(C)C)[C@@]([2H])(C(=O)N[C@]([2H])(C#N)C[C@]2([2H])CCNC2=O)N1C(=O)[C@]([2H])(NC(=O)C(F)(F)F)C(C)(C)C] |
| 29 | 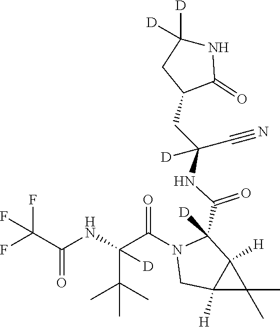 [SMILES: [2H]C1([2H])C[C@@H](C[C@@]([2H])(C#N)NC(=O)[C@]2([2H])[C@@H]3[C@H](CN2C(=O)[C@]([2H])(NC(=O)C(F)(F)F)C(C)(C)C)C3(C)C)C(=O)N1] |
| 30 | 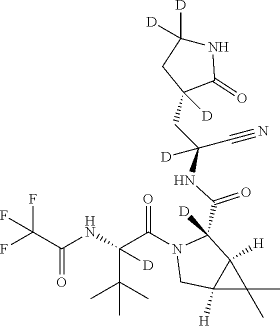 [SMILES: [2H]C1([2H])C[C@@]([2H])(C[C@@]([2H])(C#N)NC(=O)[C@]2([2H])[C@@H]3[C@H](CN2C(=O)[C@]([2H])(NC(=O)C(F)(F)F)C(C)(C)C)C3(C)C)C(=O)N1] |
| 31 | 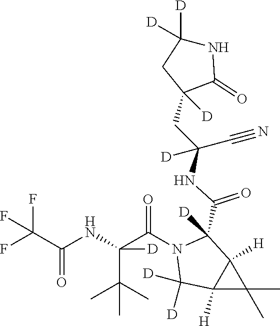 [SMILES: [2H]C1([2H])C[C@@]([2H])(C[C@@]([2H])(C#N)NC(=O)[C@]2([2H])[C@@H]3[C@@H](C3(C)C)C([2H])([2H])N2C(=O)[C@]([2H])(NC(=O)C(F)(F)F)C(C)(C)C)C(=O)N1] |
| 32 | 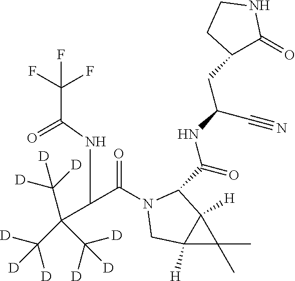 [SMILES: [2H]C([2H])([2H])C(C(NC(=O)C(F)(F)F)C(=O)N1C[C@H]2[C@@H]([C@H]1C(=O)N[C@H](C#N)C[C@@H]1CCNC1=O)C2(C)C)(C([2H])([2H])[2H])C([2H])([2H])[2H]] |
| 33 | 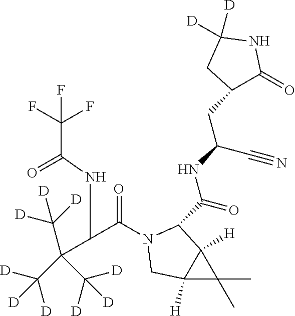 [SMILES: [2H]C1([2H])C[C@@H](C[C@@H](C#N)NC(=O)[C@@H]2[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1] |
| 34 | 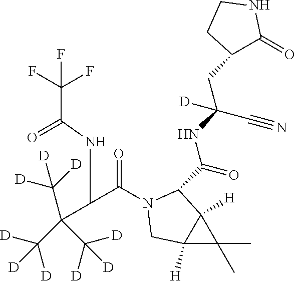 [SMILES: [2H]C([2H])([2H])C(C(NC(=O)C(F)(F)F)C(=O)N1C[C@H]2[C@@H]([C@H]1C(=O)N[C@]([2H])(C#N)C[C@@H]1CCNC1=O)C2(C)C)(C([2H])([2H])[2H])C([2H])([2H])[2H]] |
| 35 | 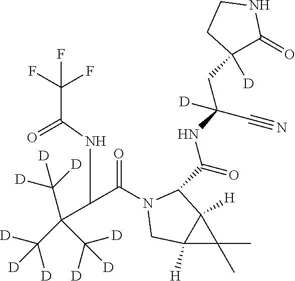 [SMILES: [2H]C([2H])([2H])C(C(NC(=O)C(F)(F)F)C(=O)N1C[C@H]2[C@@H]([C@H]1C(=O)N[C@]([2H])(C#N)C[C@]1([2H])CCNC1=O)C2(C)C)(C([2H])([2H])[2H])C([2H])([2H])[2H]] |
| 36 | 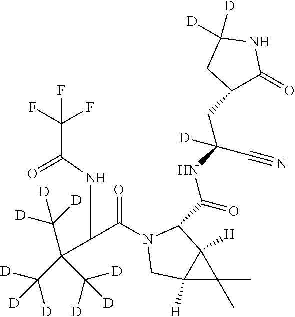 [SMILES: [2H]C1([2H])C[C@@H](C[C@@]([2H])(C#N)NC(=O)[C@@H]2[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1] |
| 37 | 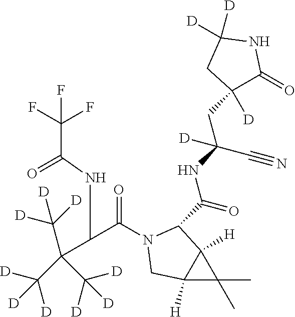 [SMILES: [2H]C1([2H])C[C@@]([2H])(C[C@@]([2H])(C#N)NC(=O)[C@@H]2[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1] |
| 38 | 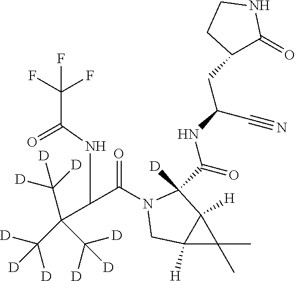 [SMILES: [2H]C([2H])([2H])C(C(NC(=O)C(F)(F)F)C(=O)N1C[C@H]2[C@H](C2(C)C)[C@@]1([2H])C(=O)N[C@H](C#N)C[C@@H]1CCNC1=O)(C([2H])([2H])[2H])C([2H])([2H])[2H]] |
| 39 | 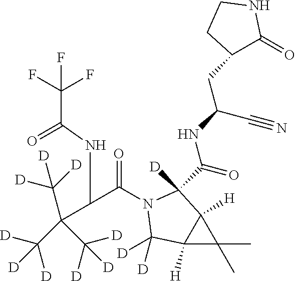 [SMILES: [2H]C1([2H])[C@H]2[C@H](C2(C)C)[C@@]([2H])(C(=O)N[C@H](C#N)C[C@@H]2CCNC2=O)N1C(=O)C(NC(=O)C(F)(F)F)C(C([2H])([2H])[2H])(C([2H])([2H])[2H])C([2H])([2H])[2H]] |
| 40 | 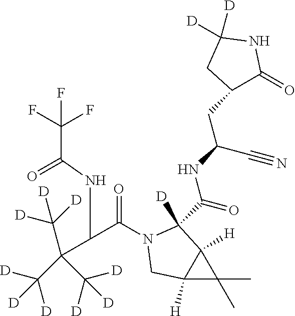 [SMILES: [2H]C1([2H])C[C@@H](C[C@@H](C#N)NC(=O)[C@]2([2H])[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1] |
| 41 | 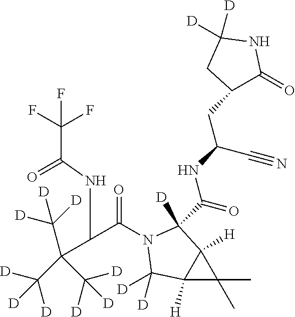 [SMILES: [2H]C1([2H])C[C@@H](C[C@@H](C#N)NC(=O)[C@]2([2H])[C@@H]3[C@@H](C3(C)C)C([2H])([2H])N2C(=O)C(NC(=O)C(F)(F)F)C(C([2H])([2H])[2H])(C([2H])([2H])[2H])C([2H])([2H])[2H])C(=O)N1] |
| 42 | 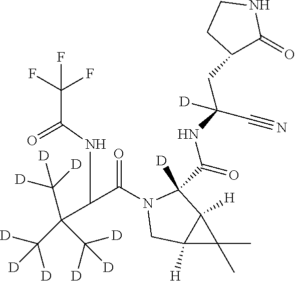 [SMILES: [2H]C([2H])([2H])C(C(NC(=O)C(F)(F)F)C(=O)N1C[C@H]2[C@H](C2(C)C)[C@@]1([2H])C(=O)N[C@]([2H])(C#N)C[C@@H]1CCNC1=O)(C([2H])([2H])[2H])C([2H])([2H])[2H]] |
| 43 | 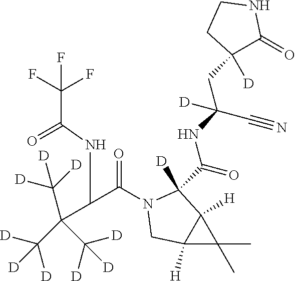 [SMILES: [2H]C([2H])([2H])C(C(NC(=O)C(F)(F)F)C(=O)N1C[C@H]2[C@H](C2(C)C)[C@@]1([2H])C(=O)N[C@]([2H])(C#N)C[C@]1([2H])CCNC1=O)(C([2H])([2H])[2H])C([2H])([2H])[2H]] |
| 44 | 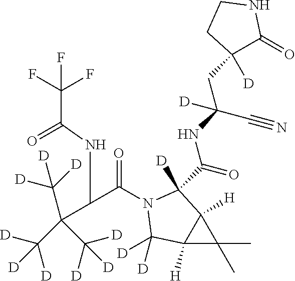 [SMILES: [2H]C1([2H])[C@H]2[C@H](C2(C)C)[C@@]([2H])(C(=O)N[C@]([2H])(C#N)C[C@]2([2H])CCNC2=O)N1C(=O)C(NC(=O)C(F)(F)F)C(C([2H])([2H])[2H])(C([2H])([2H])[2H])C([2H])([2H])[2H]] |
| 45 | 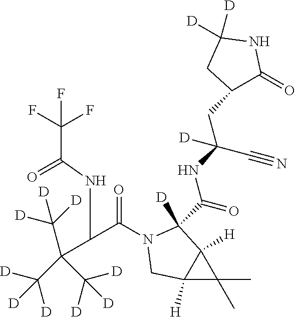 [SMILES: [2H]C1([2H])C[C@@H](C[C@@]([2H])(C#N)NC(=O)[C@]2([2H])[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1] |
| 46 | 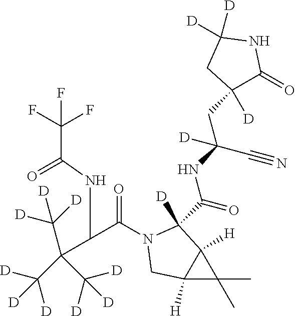 [SMILES: [2H]C1([2H])C[C@@]([2H])(C[C@@]([2H])(C#N)NC(=O)[C@]2([2H])[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1] |
| 47 | 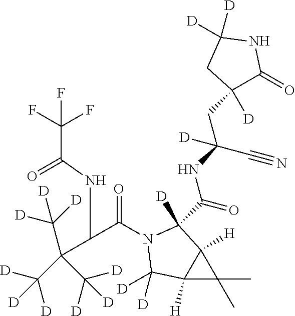 [SMILES: [2H]C1([2H])C[C@@]([2H])(C[C@@]([2H])(C#N)NC(=O)[C@]2([2H])[C@@H]3[C@@H](C3(C)C)C([2H])([2H])N2C(=O)C(NC(=O)C(F)(F)F)C(C([2H])([2H])[2H])(C([2H])([2H])[2H])C([2H])([2H])[2H])C(=O)N1] |
| 48 | 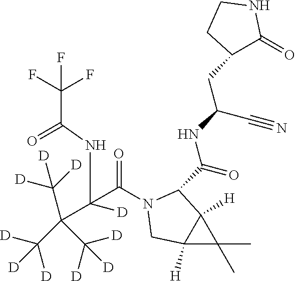 [SMILES: [2H]C([2H])([2H])C(C([2H])([2H])[2H])(C([2H])([2H])[2H])C([2H])(NC(=O)C(F)(F)F)C(=O)N1C[C@H]2[C@@H]([C@H]1C(=O)N[C@H](C#N)C[C@@H]1CCNC1=O)C2(C)C] |
| 49 | 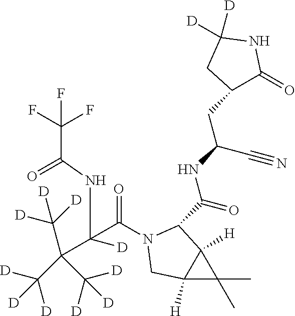 [SMILES: [2H]C1([2H])C[C@@H](C[C@@H](C#N)NC(=O)[C@@H]2[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1] |
| 50 | 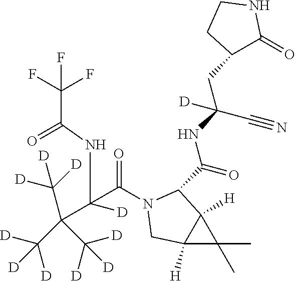 [SMILES: [2H]C([2H])([2H])C(C([2H])([2H])[2H])(C([2H])([2H])[2H])C([2H])(NC(=O)C(F)(F)F)C(=O)N1C[C@H]2[C@@H]([C@H]1C(=O)N[C@]([2H])(C#N)C[C@@H]1CCNC1=O)C2(C)C] |
| 51 | 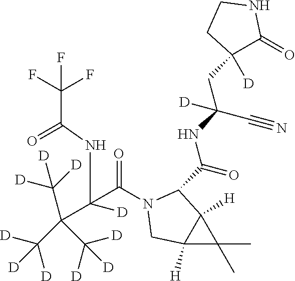 [SMILES: [2H]C([2H])([2H])C(C([2H])([2H])[2H])(C([2H])([2H])[2H])C([2H])(NC(=O)C(F)(F)F)C(=O)N1C[C@H]2[C@@H]([C@H]1C(=O)N[C@]([2H])(C#N)C[C@]1([2H])CCNC1=O)C2(C)C] |
| 52 | 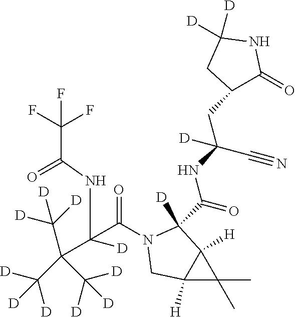 [SMILES: [2H]C1([2H])C[C@@H](C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1] |
| 53 | 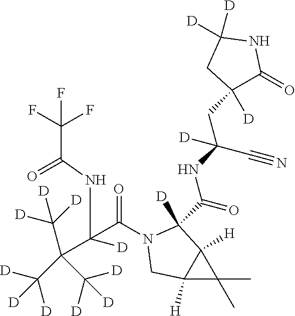 [SMILES: [2H]C1([2H])C[C@@]([2H])(C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1] |
| 54 | 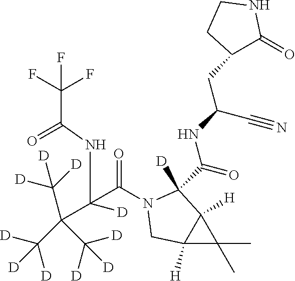 [SMILES: [2H]C([2H])([2H])C(C([2H])([2H])[2H])(C([2H])([2H])[2H])C([2H])(NC(=O)C(F)(F)F)C(=O)N1C[C@H]2[C@H](C2(C)C)[C@@]1([2H])C(=O)N[C@H](C#N)C[C@@H]1CCNC1=O] |
| 55 | 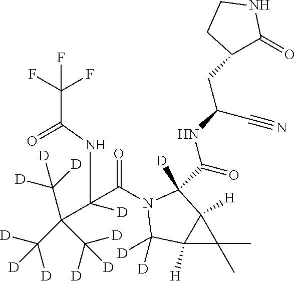 [SMILES: [2H]C1([2H])[C@H]2[C@H](C2(C)C)[C@@]([2H])(C(=O)N[C@H](C#N)C[C@@H]2CCNC2=O)N1C(=O)C([2H])(NC(=O)C(F)(F)F)C(C([2H])([2H])[2H])(C([2H])([2H])[2H])C([2H])([2H])[2H]] |
| 56 | 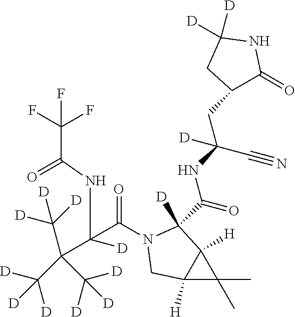 [SMILES: [2H]C1([2H])C[C@@H](C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1] |
| 57 | 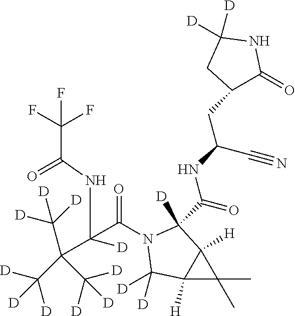 [SMILES: [2H]C1([2H])C[C@@H](C[C@@H](C#N)NC(=O)[C@]2([2H])[C@@H]3[C@@H](C3(C)C)C([2H])([2H])N2C(=O)C([2H])(NC(=O)C(F)(F)F)C(C([2H])([2H])[2H])(C([2H])([2H])[2H])C([2H])([2H])[2H])C(=O)N1] |
| 58 | 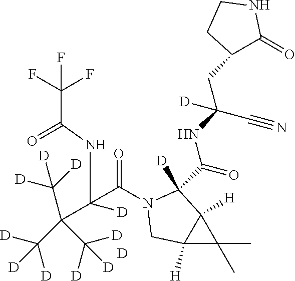 |
| 59 | 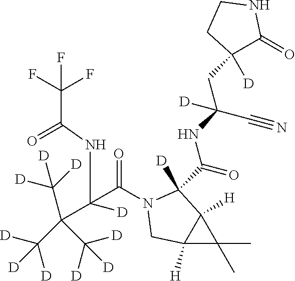 [SMILES: [2H]C([2H])([2H])C(C([2H])([2H])[2H])(C([2H])([2H])[2H])C([2H])(NC(=O)C(F)(F)F)C(=O)N1C[C@H]2[C@H](C2(C)C)[C@@]1([2H])C(=O)N[C@]([2H])(C#N)C[C@]1([2H])CCNC1=O] |
| 60 | 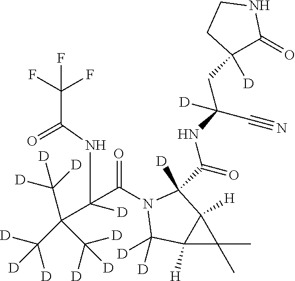 [SMILES: [2H]C1([2H])[C@H]2[C@H](C2(C)C)[C@@]([2H])(C(=O)N[C@]([2H])(C#N)C[C@]2([2H])CCNC2=O)N1C(=O)C([2H])(NC(=O)C(F)(F)F)C(C([2H])([2H])[2H])(C([2H])([2H])[2H])C([2H])([2H])[2H]] |
| 61 | 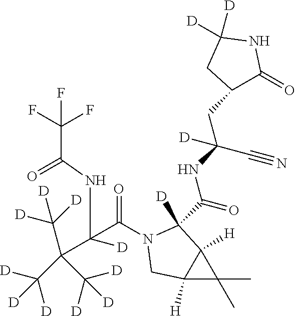 [SMILES: [2H]C1([2H])C[C@@H](C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1] |
| 62 | 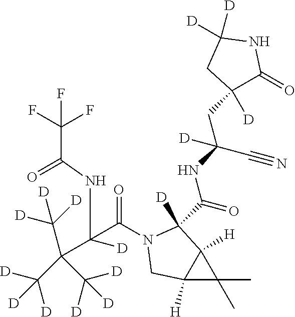 [SMILES: [2H]C1([2H])C[C@@]([2H])(C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1] |
| 63 | 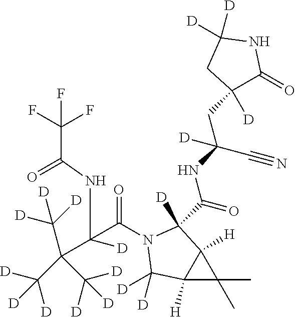 [SMILES: [2H]C1([2H])C[C@@]([2H])(C[C@@]([2H])(C#N)NC(=O)[C@]2([2H])[C@@H]3[C@@H](C3(C)C)C([2H])([2H])N2C(=O)C([2H])(NC(=O)C(F)(F)F)C(C([2H])([2H])[2H])(C([2H])([2H])[2H])C([2H])([2H])[2H])C(=O)N1] |

In certain embodiments, the invention is directed to pharmaceutical compositions comprising a pharmaceutically acceptable excipient and a compound disclosed herein. In one embodiment, the pharmaceutical composition is in the form of a tablet; a capsule; a pill; or an aqueous solution, which may be a buffered solution, such as saline or phosphate buffers.

In certain embodiments, the invention is directed to the use of the compounds described herein to prepare a medicament for the treatment or prevention of a viral infection, such as human coronavirus, novel coronavirus (SARS-CoV-2), SARS coronavirus, or MERS coronavirus.

In certain embodiments, the invention is directed to a method of preparing the cyano compounds.

The novel deuterated cyano compounds exhibit better pharmacokinetic properties, better therapeutic effects, and better drug-forming properties than PF-07321332 on the basis of comparable activity as a viral inhibitor. Also disclosed are methods of preparing the compounds, pharmaceutical compositions containing the compounds, and applications using the compounds to treat a viral infection. The methods of making the compounds can be scaled up, and the compounds have good clinical value.

### EXAMPLES

The following detailed descriptions are exemplary and explanatory only and not limiting.

In the following examples, all solvents and reagents used are commercially available and used as is, unless otherwise indicated.

The procedures described below can be used to synthesize compounds 1 to 63.

The following abbreviations are used herein:
BOP: benzotriazol-1-yloxy-tris (dimethylamino) phosphonium hexafluorophosphate
(Boc)₂O: di-tert-butyl dicarbonate
DCM: dichloromethane
D₂O: heavy water
EA: ethyl acetate
EDCI: 1-(3-Dimethylaminopropyl)-3-ethylcarbodiimide
HCl/EA: ethyl hydroacetate solution
HOBt: 1-hydroxybenzotriazole
LiHMDS: lithium bis(trimethylsilyl) amine
MeOH: methanol
MTBE: methyl tert-butyl ether
NaOH: sodium hydroxide
NH₃/MeOH: ammonia in methanol
Ni: raney Nickel
NMM: N-Methylmorpholine
SM1: L-Glutamic acid
SOCl₂: dichlorosulfoxide
TEA: triethylamine
THF: tetrahydrofuran
Ru/C: ruthenium carbon

### Example 1

### Synthesis of(S)-2-Amino-3-((S)-2-carbonylpyrrolidin-3-yl) propionitrile Hydrochloride (Compound H)

### Representative Route

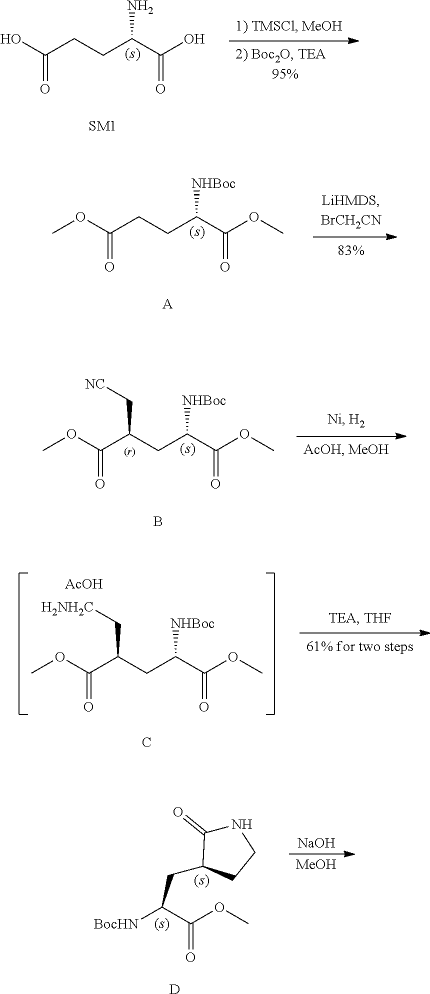

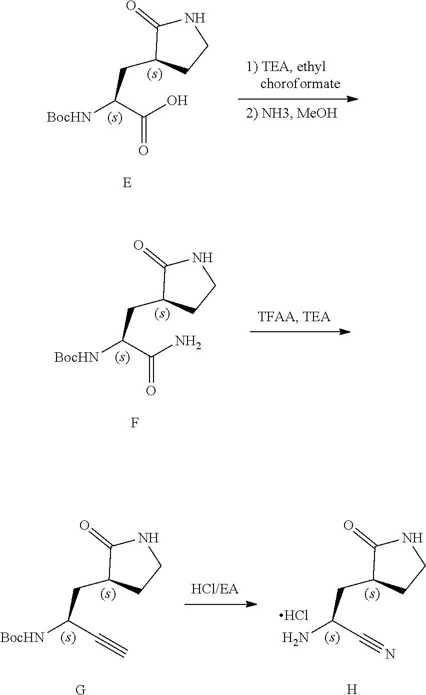

### Preparation of Intermediate Compound A

A solution of L-glutamic acid (SM1:100 g, 0.68 mol) in MeOH (500 mL) was cooled to about −5 to about 5° C., and SOCl₂ (202 g, 1.70 mol) was added dropwise. After addition, the temperature was raised to reflux. Then the solution was stirred for reaction. After completion of the reaction as indicated by TLC, the reaction mixture was concentrated to dryness. The resulting crude product was dissolved in MeOH (1 L), and TEA (172 g, 1.70 mol) was added dropwise. The resulting solution was cooled to about −5 to about 5° C., and (Boc)₂O (148.4 g, 1 eq) was added dropwise. The resulting solution was stirred at about 50 to about 60° C. for about 3 to about 5 hours and then concentrated to dryness. The resulting residue was diluted with saturated sodium bicarbonate (500 mL), then extracted 3 times with MTBE (1 L, 0.5 L, and 0.5 L). The combined organic phase was dried with anhydrous sodium sulfate, and concentrated under vacuum to provide 178 g of compound A as pale yellow oil in a 95% yield. LC-MS (ESI, m/z, C₁₂H₂₁NO₆, 276, M+H).

¹H NMR (400 MHz, CDCl₃) δ: 5.40-5.37 (m, 1H), 4.35-4.30 (m, 1H), 3.74 (s, 3H), 3.68 (s, 3H), 2.47-2.40 (m, 2H), 2.17-2.16 (m, 2H), 1.99-1.94 (m, 1H), 1.44 (s, 9H).

### Preparation of Intermediate Compound B

A solution of Compound A (150 g, 0.545 mol) in THF (450 mL) was cooled to −78° C. LiHMDS (1 M, 1.2 L, 1.2 mol) was added dropwise at controlled temperature. After addition, the reaction solution was stirred for 2 hours in a state of thermal insulation. Bromoacetonitrile (98 g, 0.817 mol) was then added dropwise at the above temperature, and the reaction was stirred for 4 hours. After completion of the reaction as indicated by TLC, the reaction solution was quenched by the dropwise addition of a solution of tetrahydrofuran in acetic acid, washed with saturated sodium chloride aqueous solution, and the organic phase dried with anhydrous sodium sulfate and concentrated to obtain crude compound B, which was purified by column chromatography (PE:EA=1:1) to obtain compound B as a pale yellow oil (142 g) in 95% yield. LC-MS (ESI, m/z, C₁₄H₂₂N₂O₆, 315, M+H).

¹H NMR (400 MHz, CDCl₃) δ: 5.19-5.17 (m, 1H), 4.38-4.33 (m, 1H), 3.77 (s, 6H), 2.88-2.79 (m, 3H), 2.19-2.14 (m, 2H), 1.45 (s, 9H).

### Preparation of Intermediate Compound D

To a solution of Compound B (50 g, 0.159 mol) and glacial acetic acid (150 mL) in MeOH (150 mL) was added Raney nickel (10 g) in a hydrogenation cylinder. The gas in the hydrogenation cylinder was replaced with hydrogen gas, and the hydrogen gas maintained at 50-60 psi. The resulting reaction mixture was stirred, maintaining heat and pressure for 4 hours. After completion of the reaction as indicated by TLC, the hydrogen was replaced with nitrogen and the resulting mixture filtered and the filtrate concentrated to dryness. The resulting crude product was dissolved in THF (500 mL), TEA (100 mL) added, and the resulting reaction solution heated to 50-60° C. and stirred for 16 hours. After completion of the reaction as indicated by TLC, the reaction solution was diluted with water (150 mL). The aqueous phase was separated and extracted twice with DCM. The obtained organic phases were combined, dried, and concentrated to afford compound D as a crude oil. The crude oil was purified by column chromatography to yield 27.8 g compound D as a white solid powder in 61% yield. LC-MS (ESI, m/z, C₁₃H₂₂N₂O₅, 287, M+H).

¹H NMR (400 MHz, CDCl₃) δ: 6.35 (s, 1H), 5.54-5.52 (m, 1H), 4.32-4.30 (m, 1H), 3.74 (s, 3H), 3.37-3.33 (m, 2H), 2.50-2.46 (m, 2H), 2.17-2.13 (m, 1H), 1.87-1.81 (m, 2H), 1.44 (s, 9H).

### Preparation of Intermediate Compound E

To a solution of Compound D (27 g, 0.094 mol) in MeOH (270 mL) was added NaOH (10 g, 0.25 mol) in a reaction flask and the resulting reaction mixture stirred for 4 hours. After the reaction was completed, the reaction mixture was concentrated and replaced with EA (270 mL), and water (100 mL) was added. The pH of the reaction solution was adjusted to 5-6 with 1N hydrochloric acid solution at 0-10° C., the aqueous phase extracted twice with EA, and the combined organic phases concentrated to provide 25 g of compound E as a white solid powder with a yield of 97%. LC-MS (ESI, m/z, C₁₃H₂₀N₂O₅, 273, M+H).

¹H NMR (400 MHz, CDCl3) δ: 6.37 (s, 1H), 5.58-5.53 (m, 1H), 4.52-4.50 (m, 1H), 3.38-3.35 (m, 2H), 2.53-2.48 (m, 2H), 2.19-2.16 (m, 1H), 1.88-1.84 (m, 2H), 1.45 (s, 9H).

### Preparation of Intermediate Compound F

To a solution of compound E (25.0 g, 0.092 mol) in THF (250 mL) was added TEA (10.2 g, 0.10 mol) in a reaction flask. The resulting reaction solution was stirred for 30 minutes and cooled to about −5 to about 5° C. Ethyl chloroformate (13.0 g, 0.12 mol) was then added dropwise. After addition, the reaction solution was stirred for 1 hour, and then NH₃/MeOH (10 M, 100 mL) was added dropwise at a temperature of about −5 to about 5° C. After addition, the resulting reaction solution was warmed to room temperature with stirring for another 2 hours. After completion of the reaction as indicated by TLC, the reaction solution was concentrated and replaced with EA (250 mL) and washed with saturated sodium chloride aqueous solution. The organic phase was then dried over anhydrous sodium sulfate, filtered, and concentrated to afford compound F as a white solid in 92% yield. LC-MS (ESI, m/z, C₁₂H₂₁N₃O₄, 272, M+H).

### Preparation of Intermediate Compound G

To a solution of compound F (23.0 g, 0.085 mol) and TEA (38.6 g, 0.382 mol) in DCM (230 mL) was added TFAA (10.2 g, 0.10 mol) in a reaction flask at about −5 to about 5° C. After addition, the resulting reaction solution was warmed to room temperature and stirred for 12 hours. After completion of the reaction as indicated by TLC, the reaction solution was washed with water and saturated sodium chloride aqueous solution successively, dried with anhydrous sodium sulfate, filtered, and concentrated to afford compound G (18.2 g) as a white solid powder in 85% yield. LC-MS (ESI, m/z, C₁₃H₂₀N₂O₃, 253, M+H).

### Preparation of Intermediate Compound H

Compound G (18.2 g, 0.072 mol) was added to a 4 mol/L solution of ethyl acetate hydrochloride (150 mL) at a temperature of 0-10° C. After stirring for 1 hour, the reaction solution was warmed to room temperature and stirred for 2 hours. After completion of the reaction as indicated by TLC, the reaction solution was concentrated to dryness to afford compound H (13.0 g) as a white solid in 95% yield. LC-MS (ESI, m/z, C₇H₁₂ClN₃O, 154, M(free base)+H).

¹H NMR (400 MHz, DMSO) δ: 8.42 (s, 2H), 8.25 (s, 1H), 7.83 (s, 1H) 3.51-3.40 (m, 3H), 2.15-1.84 (m, 5H).

### Example 2

### (S)-Synthesis of 2-Amino-3-((S)-2-carbonylpyrrolidin-3-yl-5,5-dideuterated) Propionitrile Hydrochloride (Compound H-2D1)

### Representative Route

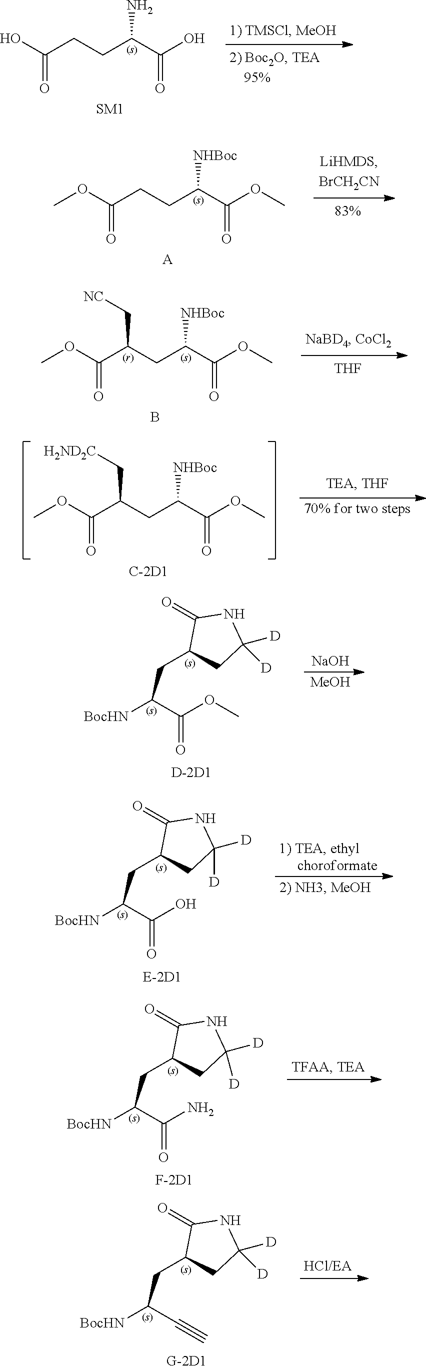

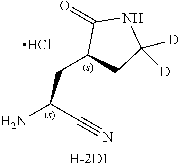

Refer to Example 1 for the preparation of Compound B.

To a solution of compound B (50 g, 0.159 mol) and cobalt chloride (20.6 g) in THF (500 mL) in a reaction flask at 0-10° C. was added sodium borodeuteride (6.7 g, 0.16 mol) in portions. After addition, the reaction was warmed to room temperature and stirred for 4 hours. After completion of the reaction as indicated by TLC, the reaction was quenched with water and dilute hydrochloric acid at about −5 to about 5° C. and the aqueous phase extracted with EA. The combined organic phases were concentrated and replaced with THF (500 mL) and TEA (100 mL) was added. The resulting reaction solution was heated to 50-60° C. and stirred for 16 hours. After completion of the reaction as indicated by TLC, the reaction solution was diluted with water (150 mL) and the layers separated. The aqueous phase was extracted twice with DCM and the organic phases combined and concentrated to dryness to afford compound D-2D1 as crude oil. The crude oil was purified by column chromatography to afford compound D-2D1 (32.1 g) as white powder in 70% yield. LC-MS (ESI, m/z, C₁₃H₂₀D₂N₂O₅, 289, M+H).

Refer to Example 1 for the preparation of subsequent intermediates.

Compound H-2D1:15.3 g was obtained. LC-MS (ESI, m/z, C₇H₁₀D₂ClN₃O, 156, M(free base)+H).

¹H NMR (400 MHz, DMSO) δ: 8.42 (s, 2H), 8.26 (s, 1H), 7.82 (s, 1H), 3.56 (m, 1H), 2.19-1.92 (m, 5H).

### Example 3

### Synthesis of (s)-2-Amino-3-((s)-2-carbonylpyrrolidin-3-yl) propionitrile-2-deuterated Hydrochloride (Compound H-1D)

### Representative Route

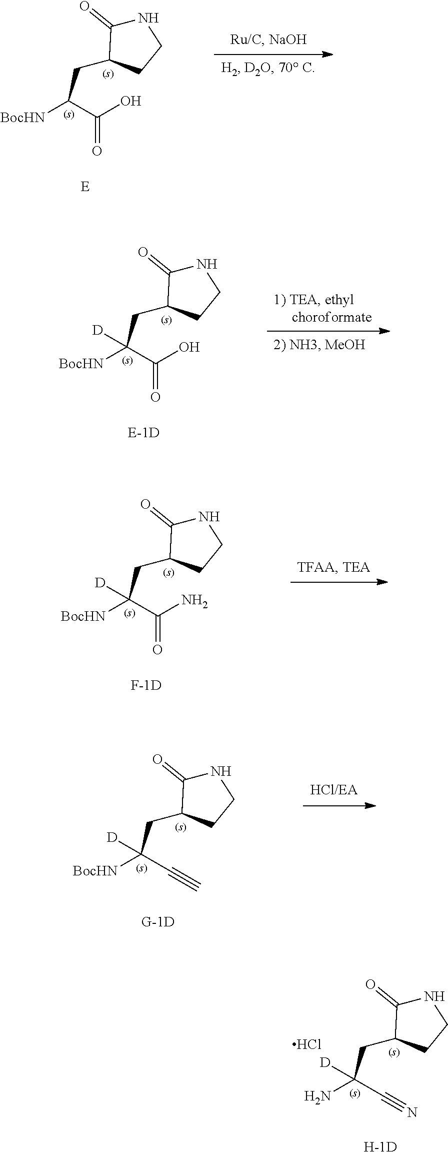

Refer to Example 1 for the preparation of Intermediate Compound E.

### Preparation of Intermediate Compound E-1D.

To a solution of Compound E (10 g, 36.7 mmol) and NaOH (4.4 g, 0.11 mol) in heavy water (100 mL) was added Ru/C (0.5 g, 5 wt %) in a hydrogenation flask. Hydrogen was exchanged 3 times and the reaction was stirred at 0.1-0.12 MPa and 70-75° C. for 52 hours. The reaction was monitored by HNMR until the reaction was indicated to be completed. The reaction mixture was then cooled and filtered. The pH of the resulting filtrate was adjusted to 5-6 and the filtrate extracted three times with EA. The combined organic phases were concentrated to dryness to afford compound E-1D (9.2 g) in a yield of 92%. LC-MS (ESI, m/z, C₁₂H₁₉DN₂O₅, 274, M+H).

Refer to Example 1 for the preparation of subsequent intermediates.

Obtain Compound H-1D: 4.8 g. LC-MS (ESI, m/z, C₇H₁₁DClN₃O, 155.2, M(free base)+H).

¹H NMR (400 MHz, DMSO) δ: 8.43 (s, 2H), 8.27 (s, 1H), 7.82 (s, 1H), 3.53-3.44 (m, 2H), 2.20-1.92 (m, 5H).

### Example 4

### Synthesis of (s)-2-Amino-3-((s)-2-carbonylpyrrolidin-3-yl-3-deuterated) propionitrile-2-deuterated Hydrochloride (Compound H-2D2)

### Representative Route.

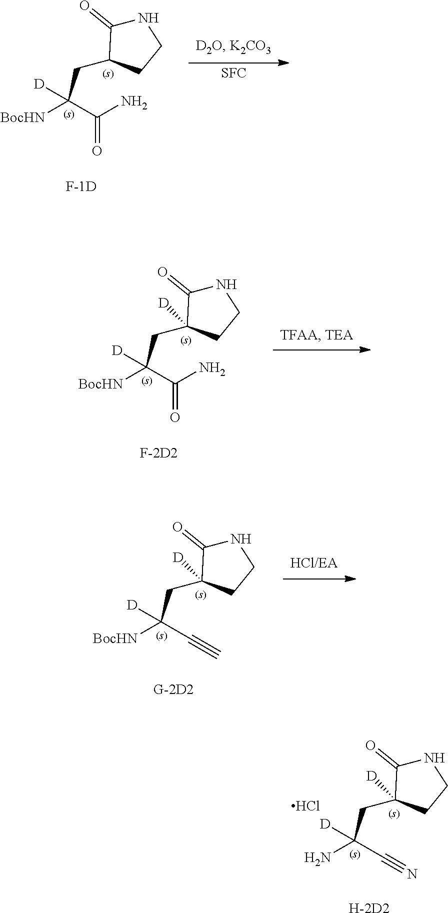

Refer to Example 1 for the preparation of intermediate compound F-1D.

### Preparation of Intermediate Compound F-2D2.

To a solution of compound F-1D (13.6 g, 0.05 mol) in heavy water (150 mL) in a reaction flask was added potassium carbonate (20.7 g, 0.15 mol). The resulting reaction mixture was stirred at 80° C. for 48 hours, extracted with EA, the organic phase dried with anhydrous sodium sulfate. Then the organic phase was purified by supercritical fluid chromatography (SFC), and concentrated to dryness to afford compound F-2D2 (6.3 g) as a white solid in 46% yield. LC-MS (ESI, m/z, C₁₂H₁₉D₂N₃O₄, 274, M+H).

Refer to Example 1 for the preparation of subsequent intermediates.

Compound H-2D2 was obtained: 3.6 g. LC-MS (ESI, m/z, C₇H₁₀D₂ClN₃O, 156, M(free base)+H).

¹H NMR (400 MHz, DMSO) δ: 8.42 (s, 2H), 8.26 (s, 1H), 7.82 (s, 1H), 3.53-3.40 (m, 2H), 2.20-1.95 (m, 4H).

### Example 5

### Synthesis of (s)-2-Amino-3-((s)-2-carbonylpyrrolidin-3-yl-3-deuterated-5,5-dideuterated) propionitrile-2-deuterated Hydrochloride (Compound H-4D)

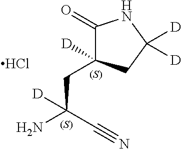

Chemical Formula: C₇H₈D₄ClN₃O. Molecular Weight: 193.67.

Compound H-4D can be synthesized according to representative routes described in Examples 2 and 4. LC-MS (ESI, m/z, C₇H₈D₄ClN₃O, 158, M(free base)+H).

¹H NMR (400 MHz, DMSO) δ: 8.43 (s, 2H), 8.24 (s, 1H), 7.81 (s, 1H), 2.20-1.94 (m, 4H).

### Example 6

### Synthesis of(S)-3,3-dimethyl-2-(2,2,2-trifluoroacetamide) butanoic Acid (K)

### Representative Route.

A solution of methyl trifluoroacetate (10.2 g, 80 mmol) in anhydrous tetrahydrofuran was

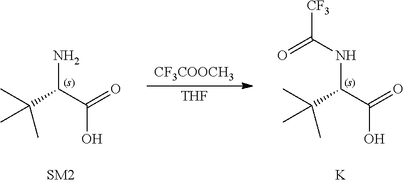

added to a reaction flask and cooled to about −5 to about 5° C. After the mixture was stirred for 15 minutes at controlled temperature, L-tertleucine (10 g, 76.2 mmol) was added. The resulting reaction solution was stirred for 1 hour at control temperature. After completion of the reaction as indicated by TLC, the reaction mixture was concentrated to provide crude compound K, which was purified by column chromatography to afford compound K (15.6 g) as white solid powder in 90% yield. LC-MS (ESI, m/z, C₈H₁₂F₃NO₃, 228, M+H).

¹H NMR (400 MHz, DMSO) δ: 12.22 (s, 1H), 8.30 (s, 1H), 4.09 (s, 1H), 0.92 (s, 9H).

### Example 7

### Synthesis of(S)-3,3-dimethyl-2-(2,2,2-trifluoroacetamide)-2-deutero-butyric Acid (K-1D)

### Representative Route

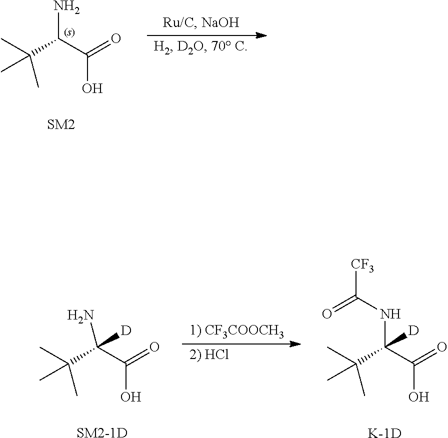

### Preparation of Intermediate Compound SM2-1D.

To a solution of L-tertleucine (10 g, 76.2 mmol) and NaOH (4.4 g, 0.11 mol) in heavy water (100 mL) was added Ru/C (0.5 g, 5 wt %) in a hydrogenation flask. Hydrogen was exchanged 3 times and the reaction mixture stirred at 0.1-0.12 MPa and 70-75° C. for 52 hours. The reaction was monitored by HNMR until the reaction was indicated to be complete. The reaction mixture was then cooled and filtered. The pH of the resulting filtrate was adjusted to 5-6, and the filtrate was extracted three times with EA. The combined organic phases were concentrated to dryness to afford compound SM2-1D (9.5 g) in a yield of 94%. LC-MS (ESI, m/z, C₆H₁₂DNO₂, 133, M+H).

Preparation of Intermediate compound K-1D.

A solution of methyl trifluoroacetate (9.6 g, 75.4 mmol) in anhydrous tetrahydrofuran was added to a reaction flask and cooled to about −5 to 5° C. After 15 minutes of stirring, SM2-1D (9.5 g, 71.8 mmol) was added. The resulting reaction solution was stirred for 1 hour at controlled temperature. After completion of the reaction as indicated by TLC, diluted hydrochloric acid was added, then the mixture was concentrated to obtain crude compound K-1D. Crude compound K-1D was further purified by column chromatography to afford compound K-1D (15.1 g) as white solid powder in 92% yield. LC-MS (ESI, m/z, C₈H₁₁DF₃NO₃, 229, M+H).

¹H NMR (400 MHz, DMSO) δ: 12.18 (s, 1H), 8.14 (s, 1H), 0.91 (s, 9H).

### Example 8

### Synthesis of(S)-3,3-dideuteromethyl-2-(2,2,2-trifluoroacetamide)-4,4,4-deuterated-butyric Acid (K-9D)

### Representative Route

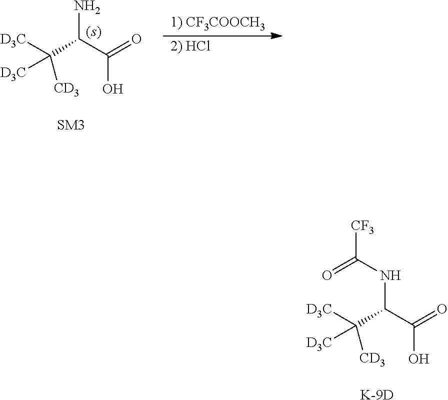

Chemical Formula: C₈H₃D₉F₃NO₃. Molecular Weight: 236.24.

Compound K-9D can be synthesized according to the representative route described in Example 6. LC-MS (ESI, m/z, C₈H₃D₉F₃NO₃, 237, M+H).

¹H NMR (400 MHz, DMSO) δ: 12.23 (s, 1H), 8.30 (s, 1H), 4.19 (s, 1H).

### Example 9

### Synthesis of(S)-3,3-dideuteromethyl-2-(2,2,2-trifluoroacetamide)-2-4,4,4-deuterated-butyric Acid (K-10D)

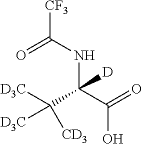

Chemical Formula: C₈H₂D₁₀F₃NO₃. Molecular Weight: 237.24.

Compound K-10D can be synthesized according to the representative route described in Example 7. LC-MS (ESI, m/z, C₈H₂D₁₀F₃NO₃, 238.1, M+H).

¹H NMR (400 MHz, DMSO) δ: 12.22 (s, 1H), 8.18 (s, 1H).

### Example 10

### Synthesis of (1R, 2S, 5S)-6,6-dimethyl-3-azabicyclo[3.1.0]hexane-2-deuterium-carboxylate (SM3-1D)

### Representative Route.

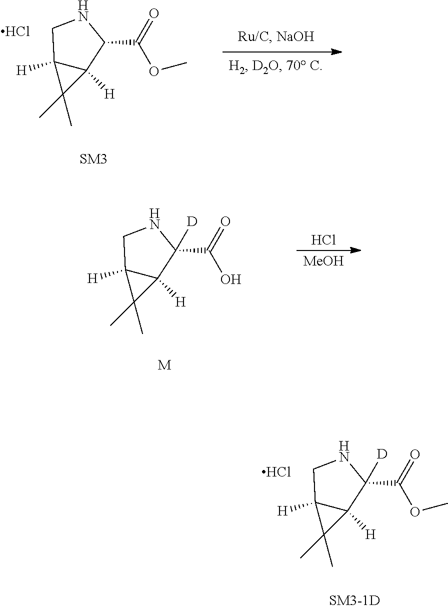

To a solution of (1R, 2S, 5S)-6,6-dimethyl-3-azabicyclo[3.1.0]hexane-carboxylate (SM3:10 g, 48.6 mmol) and NaOH (9.7 g, 0.24 mol) in heavy water (100 mL) was added Ru/C (0.5 g, 5 wt %) in a hydrogenation flask. Hydrogen was exchanged 3 times and the reaction was stirred at 0.1-0.12 MPa and 70-75° C. for 52 hours. The reaction was monitored by HNMR until the reaction was shown to be completed. The reaction mixture was then cooled and filtered. The pH of the resulting filtrate was adjusted to 5-6, and the filtrate extracted three times with EA. The combined organic phases were concentrated to dryness to afford compound M (7.0 g) in a yield of 92%.

A solution of Compound M in MeOH (100 mL) was added to a reaction flask. Hydrogen chloride gas was applied to the resulting reaction solution at 0-10° C. until the solution was saturated. The reaction was refluxed for 1 hour and warmed to room temperature. The reaction was monitored by TLC until finished and then filtered and dried to afford compound SM3-1D (8.9 g) as a white solid in 96% yield. LC-MS (ESI, m/z, C₉H₁₅DClNO₂, 171, M(free base)+H).

¹H NMR (400 MHz, DMSO) δ: 8.22 (s, 1H), 3.70 (s, 3H), 2.87-2.63 (m, 2H), 2.07 (m, 1H), 1.68 (d, 1H), 1.06-0.98 (m, 1H), 0.87 (s, 6H).

### Example 11

### Synthesis of (1R, 2S, 5S)-6,6-dimethyl-3-azabicyclo[3.1.0]hexane-2,4,4-deuterium-carboxylate (SM3-3D)

### Representative Route.

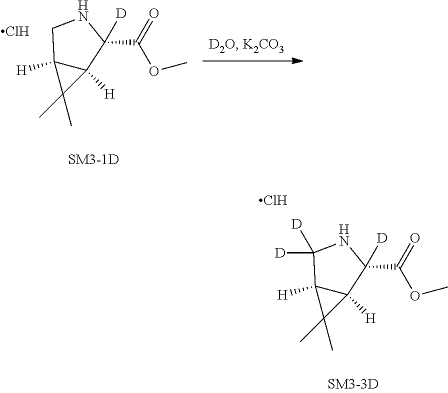

To a solution of compound SM3-1D (10 g, 48.4 mmol) in heavy water (150 mL in a reaction flask) was added potassium carbonate (31.6 g, 0.23 mol). The resulting reaction mixture was stirred at 60° C. for 48 hours, then extracted with EA. The combined organic phase was dried with anhydrous sodium sulfate, and filtered. The mixture was combined with a solution of hydrochloric acid in EA (4 mol/L, 150 mL) and stirred for 4 hours. The resulting reaction mixture was then concentrated to dryness to afford 9.3 g of compound SM3-3D in a yield of 92%. LC-MS (ESI, m/z, C₉H₁₃D₃ClNO₂, 173, M(free base)+H).

¹H NMR (400 MHz, DMSO) δ: 8.22 (s, 1H), 3.69 (s, 3H), 2.08 (s, 1H), 1.65 (d, 1H), 1.0 (d, 1H), 0.87 (s, 6H).

### Example 12

### Synthesis of Compound 1.

### Representative Route.

### Preparation of Intermediate Compound M.

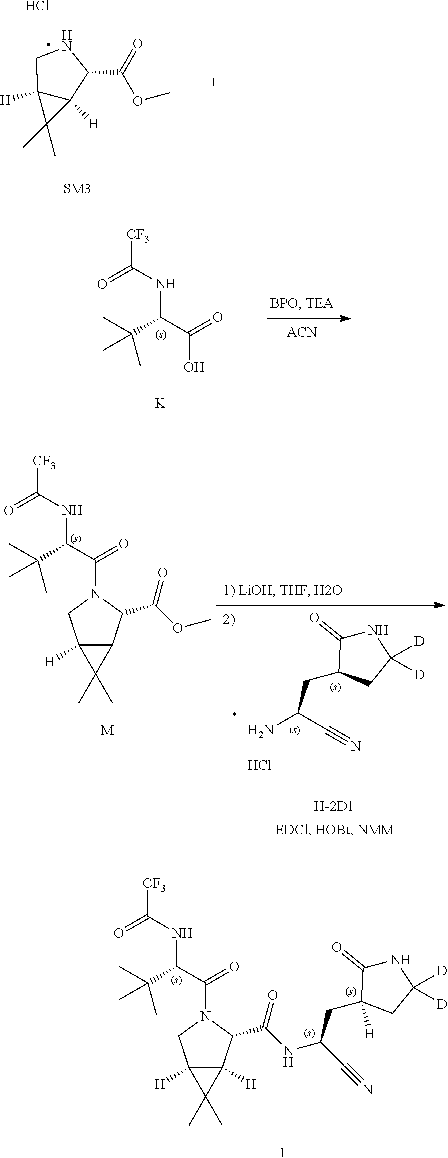

To a solution of compound K (5 g, 22 mmol) and SM3 (4.9 g, 22 mmol) in acetonitrile (100 mL) in a reaction flask were sequentially added BOP (9.7 g, 22 mmol) and TEA (4.4 g, 44 mmol). After stirring at room temperature for 2 hours, the reaction was shown by TLC to be complete. After addition of water (50 mL), the reaction was extracted three times with EA. The combined organic phases were washed with 2 mol/L HCl, 5% NaHCO₃ and water successively, dried with anhydrous magnesium sulfate, and concentrated to dryness to obtain compound M (8.0 g) as an almost white solid in 96.1% yield. LC-MS (ESI, m/z, C₁₇H₂₅F₃N₂O₄, 379, M+H).

### Preparation of Compound 1.

Compound M (8 g, 21.1 mmol) was dissolved in THF (80 mL) and MeOH (80 mL) at room temperature and an aqueous solution of lithium hydroxide (1.5 g, 62.8 mmol) (15 mL) was added. After being stirred at room temperature for 2 hours, the resulting reaction mixture was cooled to 0-10° C. Ethyl acetate was added to the mixture. Then the solution was acidified with 1N HCl, and the aqueous phase extracted twice with EA. The combined organic phases were dried over anhydrous magnesium sulfate and concentrated to dryness to afford the crude product. The crude product was further purified by column chromatography to obtain hydrolyzed compound M.

To a solution of hydrolyzed compound M (obtained in the previous step) in DMF (100 mL) in a reaction flask at about 0 to about 10° C. was added sequentially EDCI (4.9 g, 25.6 mmol), HOBt (3.4 g, 25.2 mmol), and NMM (4.3 g, 42.5 mmol). The resulting reaction mixture was stirred for 30 minutes at controlled temperature, and then H-2D1 (4.0 g, 21 mmol) was added in portions. After addition, the reaction mixture warmed to room temperature and stirred overnight. The reaction was shown to be complete by TLC. Water (80 mL) was added to the reaction mixture and the resulting mixture extracted three times with EA. The combined organic phases were washed with 0.5 mol/L HCl and 5% NaHCO₃ successively, washed with water, dried with anhydrous magnesium sulfate, and concentrated to dryness to provide crude product 1. The crude product was further purified by column chromatography to provide compound 1 (8.4 g) as an off-white solid in 79.7% yield. LC-MS (ESI, m/z, C₂₃H₃₀D₂F₃N₅O₄, 502, M+H).

¹H NMR (600 MHz, DMSO) δ: 9.44-9.41 (m, 1H), 9.05-9.02 (m, 1H), 7.68 (s, 1H), 5.0-4.94 (m, 1H), 4.43-4.40 (m, 1H), 4.15 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

Examples 13-74 below were prepared using a method similar to that described in Example 12.

### Example 13

### Synthesis of Compound 2

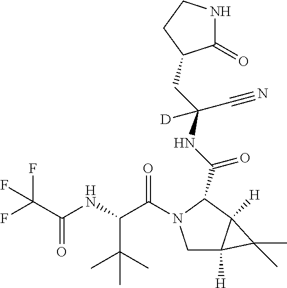 [SMILES: [2H][C@@](C#N)(C[C@@H]1CCNC1=O)NC(=O)[C@@H]1[C@@H]2[C@H](CN1C(=O)[C@@H](NC(=O)C(F)(F)F)C(C)(C)C)C2(C)C]

Chemical Formula: C₂₃H₃₁DF₃N₅O₄. Molecular Weight: 500.54. LC-MS (ESI, m/z, C₂₃H₃₁DF₃N₅O₄, 501, M+H).

¹H NMR (600 MHz, DMSO) δ: 9.44-9.40 (m, 1H), 9.05-9.01 (m, 1H), 7.67 (s, 1H), 4.43-4.41 (m, 1H), 4.15 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 14

### Synthesis of Compound 3.

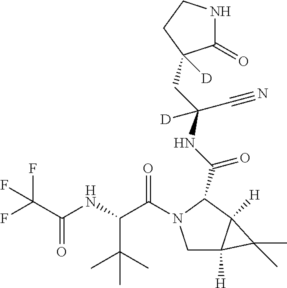 [SMILES: [2H][C@@]1(C[C@@]([2H])(C#N)NC(=O)[C@@H]2[C@@H]3[C@H](CN2C(=O)[C@@H](NC(=O)C(F)(F)F)C(C)(C)C)C3(C)C)CCNC1=O]

Chemical Formula: C₂₃H₃₀D₂F₃N₅O₄. Molecular Weight: 501.55. LC-MS (ESI, m/z, C₂₃H₃₀D₂F₃N₅O₄, 502, M+H).

¹H NMR (600 MHz, DMSO) δ: 9.44-9.41 (m, 1H), 9.06-9.02 (m, 1H), 7.69 (s, 1H), 4.44-4.40 (m, 1H), 4.16 (s, 1H), 3.93-3.90 (m, 1H), 3.71-3.68 (m, 1H), 3.14 (m, 1H), 3.05 (m, 1H), 2.18 (m, 1H), 2.10 (m, 1H), 1.73 (s, 2H), 1.57 (m, 1H), 1.33-1.30 (m, 1H), 1.03 (s, 3H), 0.98 (s, 9H), 0.85 (s, 3H).

### Example 15

### Synthesis of Compound 4.

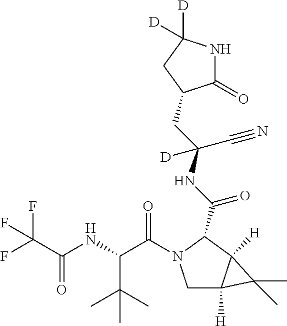 [SMILES: [2H]C1([2H])C[C@@H](C[C@@]([2H])(C#N)NC(=O)[C@@H]2[C@@H]3[C@H](CN2C(=O)[C@@H](NC(=O)C(F)(F)F)C(C)(C)C)C3(C)C)C(=O)N1]

Chemical Formula: C₂₃H₂₉D₃F₃N₅O₄. Molecular Weight: 502.55. LC-MS (ESI, m/z, C₂₃H₂₉D₃F₃N₅O₄, 503, M+H).

¹H NMR (600 MHz, DMSO): 9.44-9.41 (m, 1H), 9.05-9.01 (m, 1H), 7.68 (s, 1H), 4.44-4.41 (m, 1H), 4.15 (s, 1H), 3.94-3.91 (m, 1H), 3.71-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 16

### Synthesis of Compound 5.

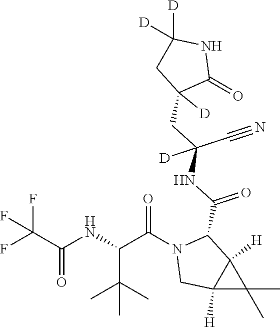 [SMILES: [2H]C1([2H])C[C@@]([2H])(C[C@@]([2H])(C#N)NC(=O)[C@@H]2[C@@H]3[C@H](CN2C(=O)[C@@H](NC(=O)C(F)(F)F)C(C)(C)C)C3(C)C)C(=O)N1]

Chemical Formula: C₂₃H₂₈D₄F₃N₅O₄. Molecular Weight: 503.56. LC-MS (ESI, m/z, C₂₃H₂₈D₄F₃N₅O₄, 504, M+H).

¹H NMR (600 MHz, DMSO): 9.44-9.40 (m, 1H), 9.05-9.02 (m, 1H), 7.68 (s, 1H), 4.44-4.40 (m, 1H), 4.15 (s, 1H), 3.94-3.90 (m, 1H), 3.72-3.68 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 17

### Synthesis of Compound 6.

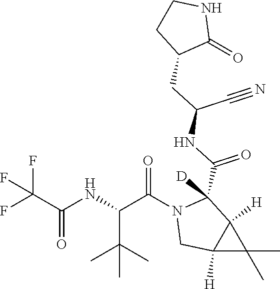 [SMILES: [2H][C@@]1(C(=O)N[C@H](C#N)C[C@@H]2CCNC2=O)[C@@H]2[C@H](CN1C(=O)[C@@H](NC(=O)C(F)(F)F)C(C)(C)C)C2(C)C]

Chemical Formula: C₂₃H₃₁DF₃N₅O₄. Molecular Weight: 500.54. LC-MS (ESI, m/z, C₂₃H₃₁DF₃N₅O₄, 501, M+H).

¹H NMR (600 MHz, DMSO) δ: 9.44-9.41 (m, 1H), 9.05-9.01 (m, 1H), 7.68 (s, 1H), 5.03-4.94 (m, 1H), 4.45-4.41 (m, 1H), 3.94-3.91 (m, 1H), 3.73-3.69 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 18

### Synthesis of Compound 7.

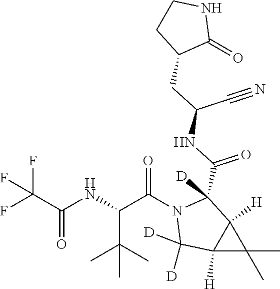 [SMILES: [2H]C1([2H])[C@H]2[C@H](C2(C)C)[C@@]([2H])(C(=O)N[C@H](C#N)C[C@@H]2CCNC2=O)N1C(=O)[C@@H](NC(=O)C(F)(F)F)C(C)(C)C]

Chemical Formula: C₂₃H₂₉D₃F₃N₅O₄. Molecular Weight: 502.55. LC-MS (ESI, m/z, C₂₃H₂₉D₃F₃N₅O₄, 503, M+H)

¹H NMR (600 MHz, DMSO) δ: 9.44-9.40 (m, 1H), 9.05-9.01 (m, 1H), 7.68 (s, 1H), 5.02-4.94 (m, 1H), 4.44-4.41 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 19

### Synthesis of Compound 8.

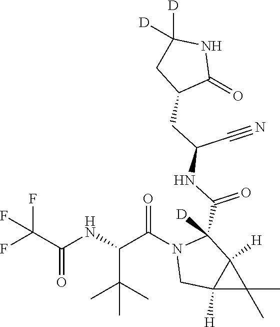 [SMILES: [2H]C1([2H])C[C@@H](C[C@@H](C#N)NC(=O)[C@]2([2H])[C@@H]3[C@H](CN2C(=O)[C@@H](NC(=O)C(F)(F)F)C(C)(C)C)C3(C)C)C(=O)N1]

Chemical Formula: C₂₃H₂₉D₃F₃N₅O₄. Molecular Weight: 502.55. LC-MS (ESI, m/z, C₂₃H₂₉D₃F₃N₅O₄, 503, M+H).

¹H NMR (600 MHz, DMSO) δ: 9.44-9.40 (m, 1H), 9.05-9.02 (m, 1H), 7.68 (s, 1H), 5.03-4.94 (m, 1H), 4.43-4.40 (m, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 20

### Synthesis of Compound 9.

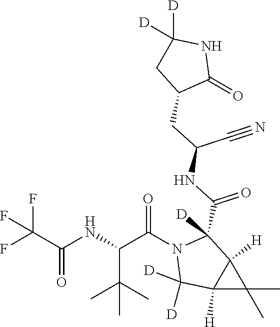 [SMILES: [2H]C1([2H])C[C@@H](C[C@@H](C#N)NC(=O)[C@]2([2H])[C@@H]3[C@@H](C3(C)C)C([2H])([2H])N2C(=O)[C@@H](NC(=O)C(F)(F)F)C(C)(C)C)C(=O)N1]

Chemical Formula: C₂₃H₂₇D₅F₃N₅O₄. Molecular Weight: 504.57. LC-MS (ESI, m/z, C₂₃H₂₇D₅F₃N₅O₄, 505, M+H).

1H NMR (400 MHz, DMSO) δ: 8.32 (s, 1H), 8.18 (s, 1H), 7.79 (s, 1H), 4.50 (m, 1H), 4.24 (s, 1H), 2.20-1.90 (m, 5H), 1.3 (d, 1H, J=8 Hz), 0.97-0.89 (m, 16H).

¹H NMR (600 MHz, DMSO) δ: 9.44-9.41 (m, 1H), 9.05-9.01 (m, 1H), 7.68 (s, 1H), 5.02-4.94 (m, 1H), 4.43-4.41 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 21

### Synthesis of Compound 10.

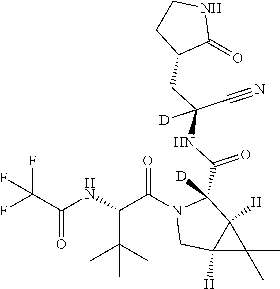 [SMILES: [2H][C@@](C#N)(C[C@@H]1CCNC1=O)NC(=O)[C@]1([2H])[C@@H]2[C@H](CN1C(=O)[C@@H](NC(=O)C(F)(F)F)C(C)(C)C)C2(C)C]

Chemical Formula: C₂₃H₃₀D₂F₃N₅O₄. Molecular Weight: 501.55. LC-MS (ESI, m/z, C₂₃H₃₀D₂F₃N₅O₄, 502, M+H).

¹H NMR (600 MHz, DMSO) δ: 9.43-9.40 (m, 1H), 9.05-9.02 (m, 1H), 7.67 (s, 1H), 4.44-4.41 (m, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 22

### Synthesis of Compound 11.

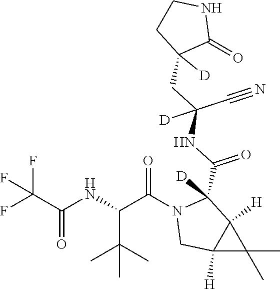 [SMILES: [2H][C@@]1(C[C@@]([2H])(C#N)NC(=O)[C@]2([2H])[C@@H]3[C@H](CN2C(=O)[C@@H](NC(=O)C(F)(F)F)C(C)(C)C)C3(C)C)CCNC1=O]

Chemical Formula: C₂₃H₂₉D₃F₃N₅O₄. Molecular Weight: 502.55. LC-MS (ESI, m/z, C₂₃H₂₉D₃F₃N₅O₄, 502, M+H).

1H NMR (600 MHz, DMSO) δ: 9.44-9.40 (m, 1H), 9.05-9.01 (m, 1H), 7.67 (s, 1H), 4.44-4.40 (m, 1H), 3.92-3.90 (m, 1H), 3.71-3.68 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 23

### Synthesis of Compound 12.

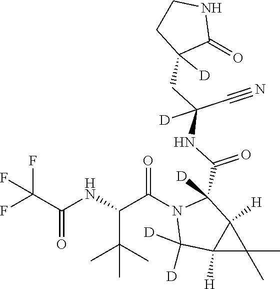 [SMILES: [2H]C1([2H])[C@H]2[C@H](C2(C)C)[C@@]([2H])(C(=O)N[C@]([2H])(C#N)C[C@]2([2H])CCNC2=O)N1C(=O)[C@@H](NC(=O)C(F)(F)F)C(C)(C)C]

Chemical Formula: C₂₃H₂₇D₅F₃N₅O₄. Molecular Weight: 504.57. LC-MS (ESI, m/z, C₂₃H₂₇D₅F₃N₅O₄, 505, M+H).

¹H NMR (400 MHz, DMSO) δ: 8.32 (s, 1H), 8.18 (s, 1H), 7.79 (s, 1H), 4.24 (s, 1H), 3.45-3.35 (m, 2H), 2.2-1.9 (m, 4H), 1.3 (d, 1H, J=8 Hz), 0.97-0.89 (m, 16H).

¹H NMR (600 MHz, DMSO) δ: 9.44-9.41 (m, 1H), 9.04-9.01 (m, 1H), 7.67 (s, 1H), 4.44-4.40 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 24

### Synthesis of Compound 13.

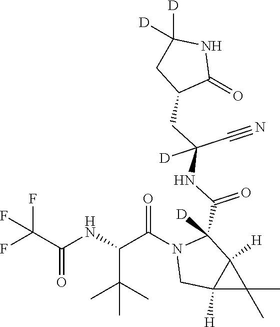 [SMILES: [2H]C1([2H])C[C@@H](C[C@@]([2H])(C#N)NC(=O)[C@]2([2H])[C@@H]3[C@H](CN2C(=O)[C@@H](NC(=O)C(F)(F)F)C(C)(C)C)C3(C)C)C(=O)N1]

Chemical Formula: C₂₃H₂₈D₄F₃N₅O₄. Molecular Weight: 503.56. LC-MS (ESI, m/z, C₂₃H₂₈D₄F₃N₅O₄, 504, M+H).

¹H NMR (600 MHz, DMSO) δ: 9.44-9.40 (m, 1H), 9.04-9.02 (m, 1H), 7.68 (s, 1H), 4.44-4.40 (m, 1H), 3.93-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 25

### Synthesis of Compound 14.

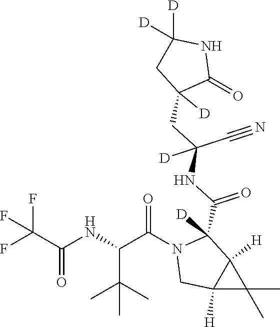 [SMILES: [2H]C1([2H])C[C@@]([2H])(C[C@@]([2H])(C#N)NC(=O)[C@]2([2H])[C@@H]3[C@H](CN2C(=O)[C@@H](NC(=O)C(F)(F)F)C(C)(C)C)C3(C)C)C(=O)N1]

Chemical Formula: C₂₃H₂₇D₅F₃N₅O₄. Molecular Weight: 504.57. LC-MS (ESI, m/z, C₂₃H₂₇D₅F₃N₅O₄, 505, M+H).

¹H NMR (600 MHz, DMSO) δ: 9.44-9.41 (m, 1H), 9.05-9.02 (m, 1H), 7.68 (s, 1H), 4.43-4.40 (m, 1H), 3.93-3.90 (m, 1H), 3.70-3.68 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 26

### Synthesis of Compound 15.

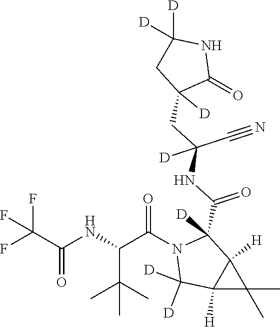 [SMILES: [2H]C1([2H])C[C@@]([2H])(C[C@@]([2H])(C#N)NC(=O)[C@]2([2H])[C@@H]3[C@@H](C3(C)C)C([2H])([2H])N2C(=O)[C@@H](NC(=O)C(F)(F)F)C(C)(C)C)C(=O)N1]

Chemical Formula: C₂₃H₂₅D₇F₃N₅O₄. Molecular Weight: 506.58. LC-MS (ESI, m/z, C₂₃H₂₅D₇F₃N₅O₄, 507, M+H).

¹H NMR (600 MHz, DMSO) δ: 9.43-9.41 (m, 1H), 9.05-9.01 (m, 1H), 7.68 (s, 1H), 4.45-4.40 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 27

### Synthesis of Compound 16.

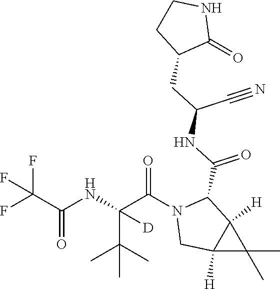 [SMILES: [2H][C@](NC(=O)C(F)(F)F)(C(=O)N1C[C@H]2[C@@H]([C@H]1C(=O)N[C@H](C#N)C[C@@H]1CCNC1=O)C2(C)C)C(C)(C)C]

Chemical Formula: C₂₃H₃₁DF₃N₅O₄. Molecular Weight: 500.54. LC-MS (ESI, m/z, C₂₃H₃₁DF₃N₅O₄, 501, M+H).

¹H NMR (600 MHz, DMSO) δ: 9.44-9.40 (m, 1H), 9.06-9.02 (m, 1H), 7.68 (s, 1H), 5.02-4.94 (m, 1H), 4.15 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 28

### Synthesis of Compound 17.

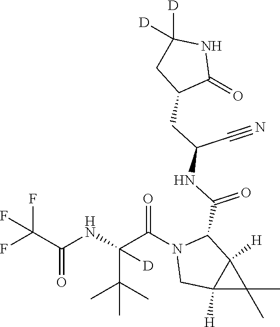 [SMILES: [2H]C1([2H])C[C@@H](C[C@@H](C#N)NC(=O)[C@@H]2[C@@H]3[C@H](CN2C(=O)[C@]([2H])(NC(=O)C(F)(F)F)C(C)(C)C)C3(C)C)C(=O)N1]

Chemical Formula: C₂₃H₂₉D₃F₃N₅O₄. Molecular Weight: 502.55. LC-MS (ESI, m/z, C₂₃H₂₉D₃F₃N₅O₄, 503, M+H).

¹H NMR (600 MHz, DMSO) δ: 9.44-9.41 (m, 1H), 9.05-9.02 (m, 1H), 7.68 (s, 1H), 5.01-4.94 (m, 1H), 4.15 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 29

### Synthesis of Compound 18.

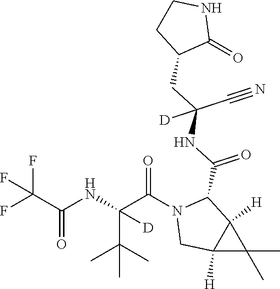 [SMILES: [2H][C@](NC(=O)C(F)(F)F)(C(=O)N1C[C@H]2[C@@H]([C@H]1C(=O)N[C@]([2H])(C#N)C[C@@H]1CCNC1=O)C2(C)C)C(C)(C)C]

Chemical Formula: C₂₃H₃₀D₂F₃N₅O₄. Molecular Weight: 501.55. LC-MS (ESI, m/z, C₂₃H₃₀D₂F₃N₅O₄, 502, M+H).

¹H NMR (600 MHz, DMSO) δ: 9.43-9.40 (m, 1H), 9.05-9.02 (m, 1H), 7.68 (s, 1H), 4.15 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 30

### Synthesis of Compound 19.

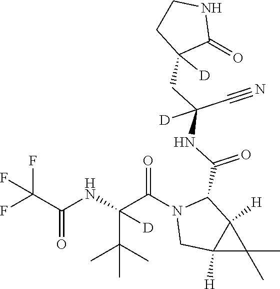 [SMILES: [2H][C@@]1(C[C@@]([2H])(C#N)NC(=O)[C@@H]2[C@@H]3[C@H](CN2C(=O)[C@]([2H])(NC(=O)C(F)(F)F)C(C)(C)C)C3(C)C)CCNC1=O]

Chemical Formula: C₂₃H₂₉D₃F₃N₅O₄. Molecular Weight: 502.55. LC-MS (ESI, m/z, C₂₃H₂₉D₃F₃N₅O₄, 503, M+H).

¹H NMR (600 MHz, DMSO) δ: 9.44-9.40 (m, 1H), 9.05-9.01 (m, 1H), 7.68 (s, 1H), 4.15 (s, 1H), 3.92-3.90 (m, 1H), 3.70-3.68 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 31

### Synthesis of Compound 20.

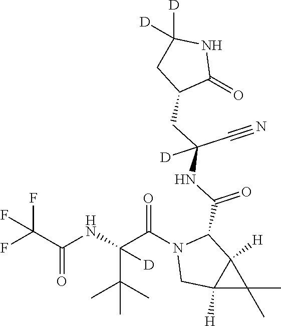 [SMILES: [2H]C1([2H])C[C@@H](C[C@@]([2H])(C#N)NC(=O)[C@@H]2[C@@H]3[C@H](CN2C(=O)[C@]([2H])(NC(=O)C(F)(F)F)C(C)(C)C)C3(C)C)C(=O)N1]

Chemical Formula: C₂₃H₂₈D₄F₃N₅O₄. Molecular Weight: 503.56. LC-MS (ESI, m/z, C₂₃H₂₈D₄F₃N₅O₄, 504, M+H).

¹H NMR (600 MHz, DMSO) δ: 9.44-9.40 (m, 1H), 9.05-9.01 (m, 1H), 7.68 (s, 1H), 4.15 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 32

### Synthesis of Compound 21.

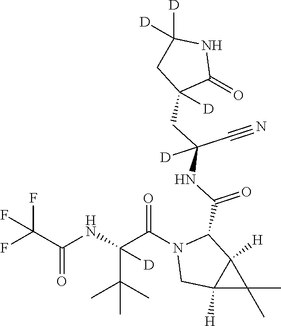 [SMILES: [2H]C1([2H])C[C@@]([2H])(C[C@@]([2H])(C#N)NC(=O)[C@@H]2[C@@H]3[C@H](CN2C(=O)[C@]([2H])(NC(=O)C(F)(F)F)C(C)(C)C)C3(C)C)C(=O)N1]

Chemical Formula: C₂₃H₂₇D₅F₃N₅O₄. Molecular Weight: 504.57. LC-MS (ESI, m/z, C₂₃H₂₇D₅F₃N₅O₄, 505, M+H).

¹H NMR (600 MHz, DMSO) δ: 9.44-9.40 (m, 1H), 9.05-9.01 (m, 1H), 7.68 (s, 1H), 4.15 (s, 1H), 3.92-3.90 (m, 1H), 3.70-3.68 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 33

### Synthesis of Compound 22.

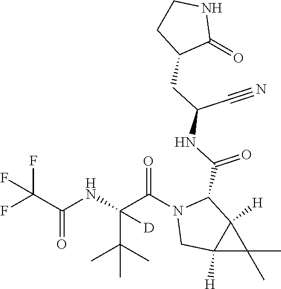 [SMILES: [2H][C@](NC(=O)C(F)(F)F)(C(=O)N1C[C@H]2[C@@H]([C@H]1C(=O)N[C@H](C#N)C[C@@H]1CCNC1=O)C2(C)C)C(C)(C)C]

Chemical Formula: C₂₃H₃₀D₂F₃N₅O₄. Molecular Weight: 501.55. LC-MS (ESI, m/z, C₂₃H₃₀D₂F₃N₅O₄, 502, M+H).

¹H NMR (600 MHz, DMSO) δ: 9.43-9.40 (m, 1H), 9.05-9.02 (m, 1H), 7.68 (s, 1H), 5.01-4.95 (m, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 34

### Synthesis of Compound 23.

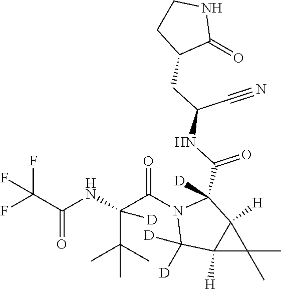 [SMILES: [2H]C1([2H])[C@H]2[C@H](C2(C)C)[C@@]([2H])(C(=O)N[C@H](C#N)C[C@@H]2CCNC2=O)N1C(=O)[C@]([2H])(NC(=O)C(F)(F)F)C(C)(C)C]

Chemical Formula: C₂₃H₂₈D₄F₃N₅O₄. Molecular Weight: 503.56. LC-MS (ESI, m/z, C₂₃H₂₈D₄F₃N₅O₄, 504, M+H).

¹H NMR (600 MHz, DMSO) δ: 9.43-9.41 (m, 1H), 9.05-9.01 (m, 1H), 7.68 (s, 1H), 5.01-4.94 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 35

### Synthesis of Compound 24.

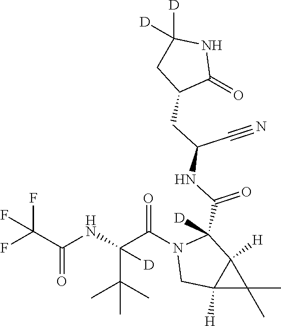 [SMILES: [2H]C1([2H])C[C@@H](C[C@@H](C#N)NC(=O)[C@]2([2H])[C@@H]3[C@H](CN2C(=O)[C@]([2H])(NC(=O)C(F)(F)F)C(C)(C)C)C3(C)C)C(=O)N1]

Chemical Formula: C₂₃H₂₈D₄F₃N₅O₄. Molecular Weight: 503.56. LC-MS (ESI, m/z, C₂₃H₂₈D₄F₃N₅O₄, 504, M+H).

¹H NMR (600 MHz, DMSO) δ: 9.44-9.40 (m, 1H), 9.05-9.02 (m, 1H), 7.68 (s, 1H), 5.01-4.94 (m, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 36

### Synthesis of Compound 25.

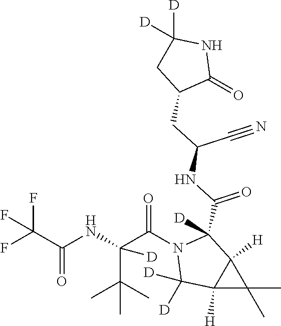 [SMILES: [2H]C1([2H])C[C@@H](C[C@@H](C#N)NC(=O)[C@]2([2H])[C@@H]3[C@@H](C3(C)C)C([2H])([2H])N2C(=O)[C@]([2H])(NC(=O)C(F)(F)F)C(C)(C)C)C(=O)N1]

Chemical Formula: C₂₃H₂₆D₆F₃N₅O₄. Molecular Weight: 505.57. LC-MS (ESI, m/z, C₂₃H₂₆D₆F₃N₅O₄, 506, M+H).

¹H NMR (600 MHz, DMSO) δ: 9.44-9.41 (m, 1H), 9.05-9.01 (m, 1H), 7.68 (s, 1H), 5.0-4.94 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 37

### Synthesis of Compound 26.

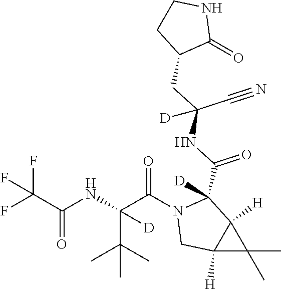 [SMILES: [2H][C@](NC(=O)C(F)(F)F)(C(=O)N1C[C@H]2[C@H](C2(C)C)[C@@]1([2H])C(=O)N[C@]([2H])(C#N)C[C@@H]1CCNC1=O)C(C)(C)C]

Chemical Formula: C₂₃H₂₉D₃F₃N₅O₄. Molecular Weight: 502.55. LC-MS (ESI, m/z, C₂₃H₂₉D₃F₃N₅O₄, 503, M+H).

¹H NMR (600 MHz, DMSO) δ: 9.43-9.41 (m, 1H), 9.05-9.02 (m, 1H), 7.68 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 38

### Synthesis of Compound 27.

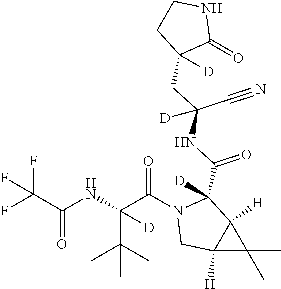 [SMILES: [2H][C@@]1(C[C@@]([2H])(C#N)NC(=O)[C@]2([2H])[C@@H]3[C@H](CN2C(=O)[C@]([2H])(NC(=O)C(F)(F)F)C(C)(C)C)C3(C)C)CCNC1=O]

Chemical Formula: C₂₃H₂₈D₄F₃N₅O₄. Molecular Weight: 503.56. LC-MS (ESI, m/z, C₂₃H₂₈D₄F₃N₅O₄, 504, M+H).

¹H NMR (600 MHz, DMSO) δ: 9.43-9.40 (m, 1H), 9.05-9.01 (m, 1H), 7.68 (s, 1H), 3.92-3.90 (m, 1H), 3.70-3.68 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 39

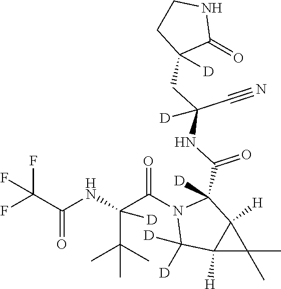 [SMILES: [2H]C1([2H])[C@H]2[C@H](C2(C)C)[C@@]([2H])(C(=O)N[C@]([2H])(C#N)C[C@]2([2H])CCNC2=O)N1C(=O)[C@]([2H])(NC(=O)C(F)(F)F)C(C)(C)C]

### Synthesis of Compound 28.

Chemical Formula: C₂₃H₂₆D₆F₃N₅O₄. Molecular Weight: 505.57. LC-MS (ESI, m/z, C₂₃H₂₆D₆F₃N₅O₄, 506, M+H).

¹H NMR (600 MHz, DMSO) δ: 9.44-9.40 (m, 1H), 9.05-9.03 (m, 1H), 7.68 (s, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 40

### Synthesis of Compound 29.

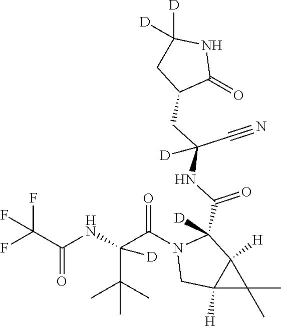 [SMILES: [2H]C1([2H])C[C@@H](C[C@@]([2H])(C#N)NC(=O)[C@]2([2H])[C@@H]3[C@H](CN2C(=O)[C@]([2H])(NC(=O)C(F)(F)F)C(C)(C)C)C3(C)C)C(=O)N1]

Chemical Formula: C₂₃H₂₇D₅F₃N₅O₄. Molecular Weight: 504.57. LC-MS (ESI, m/z, C₂₃H₂₇D₅F₃N₅O₄, 505, M+H).

¹H NMR (600 MHz, DMSO) δ: 9.44-9.41 (m, 1H), 9.05-9.02 (m, 1H), 7.68 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 41

### Synthesis of Compound 30.

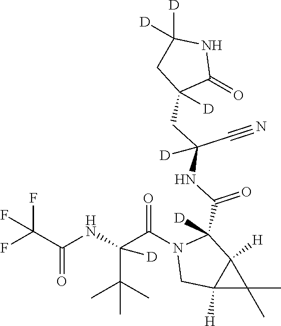 [SMILES: [2H]C1([2H])C[C@@]([2H])(C[C@@]([2H])(C#N)NC(=O)[C@]2([2H])[C@@H]3[C@H](CN2C(=O)[C@]([2H])(NC(=O)C(F)(F)F)C(C)(C)C)C3(C)C)C(=O)N1]

Chemical Formula: C₂₃H₂₆D₆F₃N₅O₄. Molecular Weight: 505.57. LC-MS (ESI, m/z, C₂₃H₂₆D₆F₃N₅O₄, 506, M+H).

¹H NMR (600 MHz, DMSO) δ: 9.44-9.40 (m, 1H), 9.06-9.02 (m, 1H), 7.68 (s, 1H), 3.93-3.90 (m, 1H), 3.70-3.68 (m, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 42

### Synthesis of Compound 31.

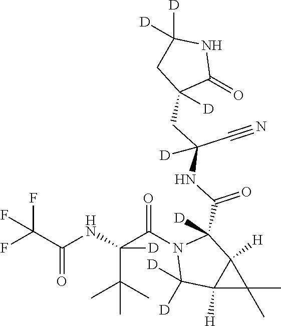 [SMILES: [2H]C1([2H])C[C@@]([2H])(C[C@@]([2H])(C#N)NC(=O)[C@]2([2H])[C@@H]3[C@@H](C3(C)C)C([2H])([2H])N2C(=O)[C@]([2H])(NC(=O)C(F)(F)F)C(C)(C)C)C(=O)N1]

Chemical Formula: C₂₃H₂₄D₈F₃N₅O₄. Molecular Weight: 507.58. LC-MS (ESI, m/z, C₂₃H₂₄D₈F₃N₅O₄, 508, M+H).

¹H NMR (600 MHz, DMSO) δ: 9.43-9.40 (m, 1H), 9.05-9.02 (m, 1H), 7.68 (s, 1H), 2.19 (m, 1H), 2.10 (m, 1H), 1.73 (s, 2H), 1.57 (m, 1H), 1.33-1.31 (m, 1H), 1.03 (s, 3H), 0.98 (s, 9H), 0.85 (s, 3H).

### Example 43

### Synthesis of Compound 32.

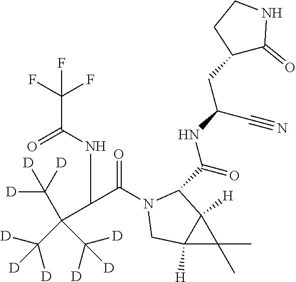 [SMILES: [2H]C([2H])([2H])C(C(NC(=O)C(F)(F)F)C(=O)N1C[C@H]2[C@@H]([C@H]1C(=O)N[C@H](C#N)C[C@@H]1CCNC1=O)C2(C)C)(C([2H])([2H])[2H])C([2H])([2H])[2H]]

Chemical Formula: C₂₃H₂₃D₉F₃N₅O₄. Molecular Weight: 508.59. LC-MS (ESI, m/z, C₂₃H₂₃D₉F₃N₅O₄, 509, M+H).

¹H NMR (600 MHz, DMSO) δ: 9.43-9.41 (m, 1H), 9.05-9.02 (m, 1H), 7.68 (s, 1H), 5.0-4.94 (m, 1H), 4.43-4.40 (m, 1H), 4.15 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 44

### Synthesis of Compound 33.

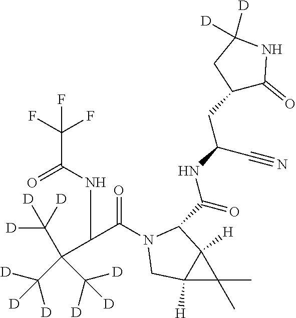 [SMILES: [2H]C1([2H])C[C@@H](C[C@@H](C#N)NC(=O)[C@@H]2[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1]

Chemical Formula: C₂₃H₂₁D₁₁F₃N₅O₄. Molecular Weight: 510.60. LC-MS (ESI, m/z, C₂₃H₂₁D₁₁F₃N₅O₄, 511, M+H).

¹H NMR (600 MHz, DMSO) δ: 9.44-9.41 (m, 1H), 9.06-9.02 (m, 1H), 7.68 (s, 1H), 5.0-4.94 (m, 1H), 4.43-4.40 (m, 1H), 4.15 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 45

### Synthesis of Compound 34.

Chemical Formula: C₂₃H₂₂D₁₀F₃N₅O₄. Molecular Weight: 509.60. LC-MS (ESI, m/z,

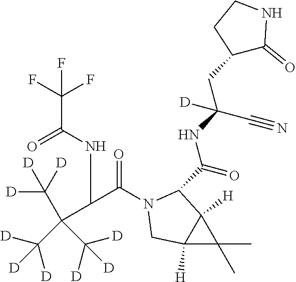 [SMILES: [2H]C([2H])([2H])C(C(NC(=O)C(F)(F)F)C(=O)N1C[C@H]2[C@@H]([C@H]1C(=O)N[C@]([2H])(C#N)C[C@@H]1CCNC1=O)C2(C)C)(C([2H])([2H])[2H])C([2H])([2H])[2H]]

C₂₃H₂₂D₁₀F₃N₅O₄, 510, M+H).

¹H NMR (600 MHz, DMSO) δ: 9.43-9.41 (m, 1H), 9.05-9.01 (m, 1H), 7.68 (s, 1H), 4.43-4.40 (m, 1H), 4.15 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 46

### Synthesis of Compound 35.

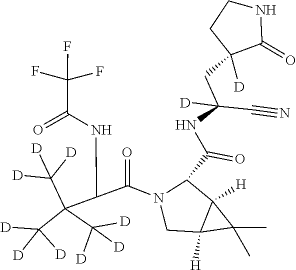 [SMILES: [2H]C([2H])([2H])C(C(NC(=O)C(F)(F)F)C(=O)N1C[C@H]2[C@@H]([C@H]1C(=O)N[C@]([2H])(C#N)C[C@]1([2H])CCNC1=O)C2(C)C)(C([2H])([2H])[2H])C([2H])([2H])[2H]]

Chemical Formula: C₂₃H₂₁D₁₁F₃N₅O₄. Molecular Weight: 510.60. LC-MS (ESI, m/z, C₂₃H₂₁D₁₁F₃N₅O₄, 511, M+H).

¹H NMR (600 MHz, DMSO) δ: 9.45-9.41 (m, 1H), 9.05-9.00 (m, 1H), 7.68 (s, 1H), 4.43-4.40 (m, 1H), 4.15 (s, 1H), 3.92-3.90 (m, 1H), 3.70-3.68 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 47

### Synthesis of Compound 36.

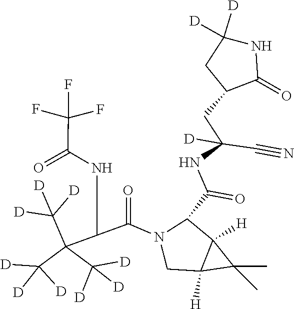 [SMILES: [2H]C1([2H])C[C@@H](C[C@@]([2H])(C#N)NC(=O)[C@@H]2[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1]

Chemical Formula: C₂₃H₂₀D₁₂F₃N₅O₄. Molecular Weight: 511.61. LC-MS (ESI, m/z, C₂₃H₂₀D₁₂F₃N₅O₄, 512, M+H).

¹H NMR (600 MHz, DMSO) δ: 9.43-9.40 (m, 1H), 9.05-9.01 (m, 1H), 7.68 (s, 1H), 4.43-4.40 (m, 1H), 4.15 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 48

### Synthesis of Compound 37.

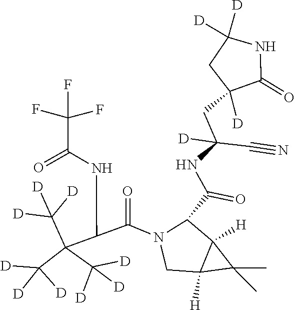 [SMILES: [2H]C1([2H])C[C@@]([2H])(C[C@@]([2H])(C#N)NC(=O)[C@@H]2[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1]

Chemical Formula: C₂₃H₁₉D₁₃F₃N₅O₄. Molecular Weight: 512.61. LC-MS (ESI, m/z, C₂₃H₁₉D₁₃F₃N₅O₄, 513, M+H).

¹H NMR (600 MHz, DMSO) δ: 9.43-9.41 (m, 1H), 9.05-9.02 (m, 1H), 7.68 (s, 1H), 4.43-4.40 (m, 1H), 4.15 (s, 1H), 3.92-3.90 (m, 1H), 3.70-3.68 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 49

### Synthesis of Compound 38.

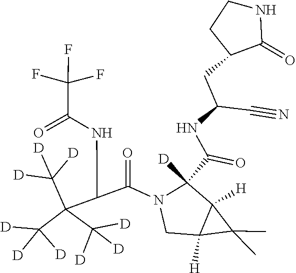 [SMILES: [2H]C([2H])([2H])C(C(NC(=O)C(F)(F)F)C(=O)N1C[C@H]2[C@H](C2(C)C)[C@@]1([2H])C(=O)N[C@H](C#N)C[C@@H]1CCNC1=O)(C([2H])([2H])[2H])C([2H])([2H])[2H]]

Chemical Formula: C₂₃H₂₂D₁₀F₃N₅O₄. Molecular Weight: 509.60. LC-MS (ESI, m/z, C₂₃H₂₂D₁₀F₃N₅O₄, 510, M+H).

¹H NMR (600 MHz, DMSO) δ: 9.44-9.41 (m, 1H), 9.05-9.02 (m, 1H), 7.68 (s, 1H), 5.0-4.94 (m, 1H), 4.43-4.40 (m, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 50

### Synthesis of Compound 39.

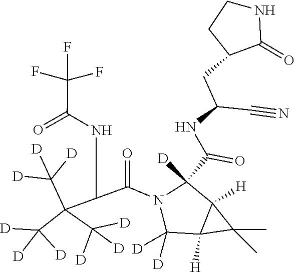 [SMILES: [2H]C1([2H])[C@H]2[C@H](C2(C)C)[C@@]([2H])(C(=O)N[C@H](C#N)C[C@@H]2CCNC2=O)N1C(=O)C(NC(=O)C(F)(F)F)C(C([2H])([2H])[2H])(C([2H])([2H])[2H])C([2H])([2H])[2H]]

Chemical Formula: C₂₃H₂₀D₁₂F₃N₅O₄. Molecular Weight: 511.61. LC-MS (ESI, m/z, C₂₃H₂₀D₁₂F₃N₅O₄, 512, M+H).

¹H NMR (600 MHz, DMSO) δ: 9.43-9.41 (m, 1H), 9.05-9.01 (m, 1H), 7.68 (s, 1H), 5.0-4.94 (m, 1H), 4.43-4.40 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 51

### Synthesis of Compound 40.

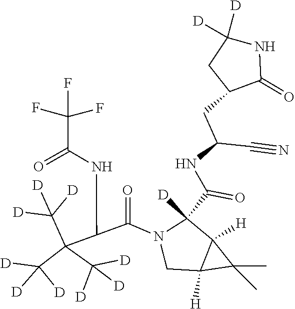 [SMILES: [2H]C1([2H])C[C@@H](C[C@@H](C#N)NC(=O)[C@]2([2H])[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1]

Chemical Formula: C₂₃H₂₀D₁₂F₃N₅O₄. Molecular Weight: 511.61. LC-MS (ESI, m/z, C₂₃H₂₀D₁₂F₃N₅O₄, 512, M+H).

¹H NMR (600 MHz, DMSO) δ: 9.44-9.41 (m, 1H), 9.05-9.02 (m, 1H), 7.68 (s, 1H), 5.0-4.94 (m, 1H), 4.43-4.40 (m, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 52

### Synthesis of Compound 41.

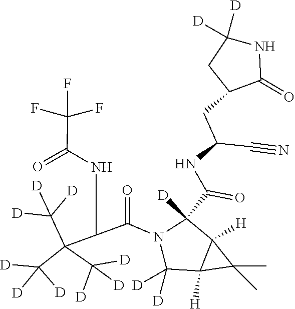 [SMILES: [2H]C1([2H])C[C@@H](C[C@@H](C#N)NC(=O)[C@]2([2H])[C@@H]3[C@@H](C3(C)C)C([2H])([2H])N2C(=O)C(NC(=O)C(F)(F)F)C(C([2H])([2H])[2H])(C([2H])([2H])[2H])C([2H])([2H])[2H])C(=O)N1]

Chemical Formula: C₂₃H₁₈D₁₄F₃N₅O₄. Molecular Weight: 513.62. LC-MS (ESI, m/z, C₂₃H₁₈D₁₄F₃N₅O₄, 514, M+H).

¹H NMR (600 MHz, DMSO) δ: 9.45-9.41 (m, 1H), 9.05-9.02 (m, 1H), 7.68 (s, 1H), 5.0-4.94 (m, 1H), 4.43-4.40 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 53

### Synthesis of Compound 42.

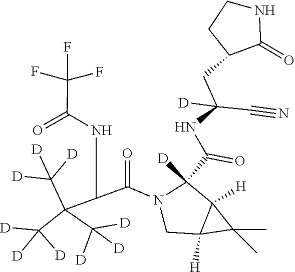 [SMILES: [2H]C([2H])([2H])C(C(NC(=O)C(F)(F)F)C(=O)N1C[C@H]2[C@H](C2(C)C)[C@@]1([2H])C(=O)N[C@]([2H])(C#N)C[C@@H]1CCNC1=O)(C([2H])([2H])[2H])C([2H])([2H])[2H]]

Chemical Formula: C₂₃H₂₁D₁₁F₃N₅O₄. Molecular Weight: 510.60. LC-MS (ESI, m/z, C₂₃H₂₁D₁₁F₃N₅O₄, 511.3, M+H).

¹H NMR (600 MHz, DMSO): 9.43-9.40 (m, 1H), 9.05-9.01 (m, 1H), 7.68 (s, 1H), 4.43-4.40 (m, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 54

### Synthesis of Compound 43.

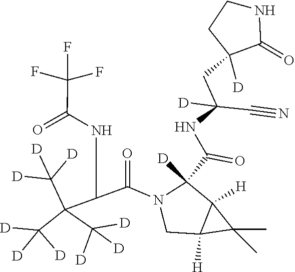 [SMILES: [2H]C([2H])([2H])C(C(NC(=O)C(F)(F)F)C(=O)N1C[C@H]2[C@H](C2(C)C)[C@@]1([2H])C(=O)N[C@]([2H])(C#N)C[C@]1([2H])CCNC1=O)(C([2H])([2H])[2H])C([2H])([2H])[2H]]

Chemical Formula: C₂₃H₂₀D₁₂F₃N₅O₄. Molecular Weight: 511.61. LC-MS (ESI, m/z, C₂₃H₂₀D₁₂F₃N₅O₄, 512, M+H).

¹H NMR (600 MHz, DMSO) δ: 9.45-9.41 (m, 1H), 9.05-9.0 (m, 1H), 7.68 (s, 1H), 4.43-4.40 (m, 1H), 3.92-3.90 (m, 1H), 3.70-3.68 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 55

### Synthesis of Compound 44.

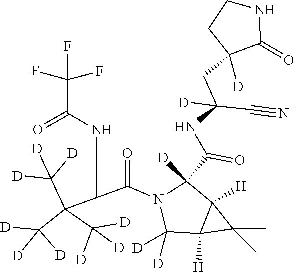 [SMILES: [2H]C1([2H])[C@H]2[C@H](C2(C)C)[C@@]([2H])(C(=O)N[C@]([2H])(C#N)C[C@]2([2H])CCNC2=O)N1C(=O)C(NC(=O)C(F)(F)F)C(C([2H])([2H])[2H])(C([2H])([2H])[2H])C([2H])([2H])[2H]]

Chemical Formula: C₂₃H₁₈D₁₄F₃N₅O₄. Molecular Weight: 513.62. LC-MS (ESI, m/z, C₂₃H₁₈D₁₄F₃N₅O₄, 514.3, M+H).

¹H NMR (600 MHz, DMSO) δ: 9.43-9.40 (m, 1H), 9.05-9.1 (m, 1H), 7.68 (s, 1H), 4.43-4.40 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 56

### Synthesis of Compound 45.

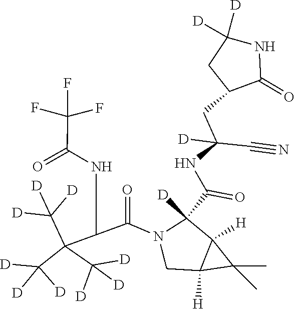 [SMILES: [2H]C1([2H])C[C@@H](C[C@@]([2H])(C#N)NC(=O)[C@]2([2H])[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1]

Chemical Formula: C₂₃H₁₉D₁₃F₃N₅O₄. Molecular Weight: 512.61. LC-MS (ESI, m/z, C₂₃H₁₉D₁₃F₃N₅O₄, 513, M+H).

¹H NMR (600 MHz, DMSO) δ: 9.45-9.41 (m, 1H), 9.06-9.02 (m, 1H), 7.68 (s, 1H), 4.43-4.40 (m, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 57

### Synthesis of Compound 46.

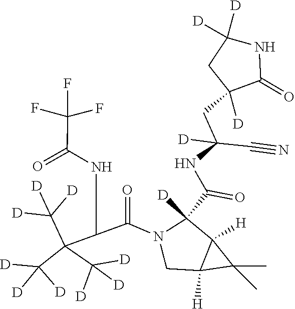 [SMILES: [2H]C1([2H])C[C@@]([2H])(C[C@@]([2H])(C#N)NC(=O)[C@]2([2H])[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1]

Chemical Formula: C₂₃H₁₈D₁₄F₃N₅O₄. Molecular Weight: 513.62. LC-MS (ESI, m/z, C₂₃H₁₈D₁₄F₃N₅O₄, 514, M+H).

¹H NMR (600 MHz, DMSO) δ: 9.44-9.41 (m, 1H), 9.05-9.02 (m, 1H), 7.68 (s, 1H), 4.43-4.40 (m, 1H), 3.92-3.90 (m, 1H), 3.70-3.68 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 58

### Synthesis of Compound 47.

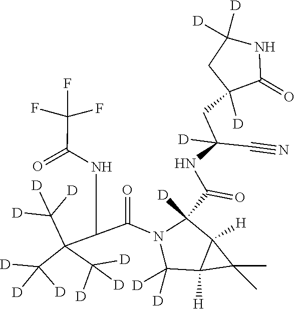 [SMILES: [2H]C1([2H])C[C@@]([2H])(C[C@@]([2H])(C#N)NC(=O)[C@]2([2H])[C@@H]3[C@@H](C3(C)C)C([2H])([2H])N2C(=O)C(NC(=O)C(F)(F)F)C(C([2H])([2H])[2H])(C([2H])([2H])[2H])C([2H])([2H])[2H])C(=O)N1]

Chemical Formula: C₂₃H₁₆D₁₆F₃N₅O₄. Molecular Weight: 515.63. LC-MS (ESI, m/z, C₂₃H₁₆D₁₆F₃N₅O₄, 516, M+H).

¹H NMR (600 MHz, DMSO) δ: 9.45-9.41 (m, 1H), 9.06-9.02 (m, 1H), 7.68 (s, 1H), 4.43-4.40 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 59

### Synthesis of Compound 48.

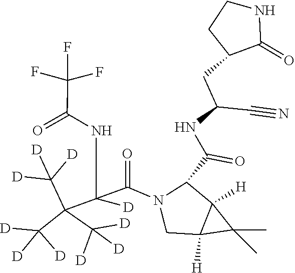 [SMILES: [2H]C([2H])([2H])C(C([2H])([2H])[2H])(C([2H])([2H])[2H])C([2H])(NC(=O)C(F)(F)F)C(=O)N1C[C@H]2[C@@H]([C@H]1C(=O)N[C@H](C#N)C[C@@H]1CCNC1=O)C2(C)C]

Chemical Formula: C₂₃H₂₂D₁₀F₃N₅O₄. Molecular Weight: 509.60. LC-MS (ESI, m/z, C₂₃H₂₂D₁₀F₃N₅O₄, 510, M+H).

¹H NMR (600 MHz, DMSO) δ: 9.44-9.41 (m, 1H), 9.05-9.02 (m, 1H), 7.68 (s, 1H), 5.0-4.94 (m, 1H), 4.15 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 60

### Synthesis of Compound 49.

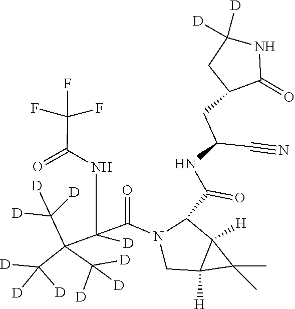 [SMILES: [2H]C1([2H])C[C@@H](C[C@@H](C#N)NC(=O)[C@@H]2[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1]

Chemical Formula: C₂₃H₂₀D₁₂F₃N₅O₄. Molecular Weight: 511.61. LC-MS (ESI, m/z, C₂₃H₂₀D₁₂F₃N₅O₄, 512, M+H).

¹H NMR (600 MHz, DMSO) δ: 9.44-9.40 (m, 1H), 9.06-9.02 (m, 1H), 7.68 (s, 1H), 5.0-4.94 (m, 1H), 4.15 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 61

### Synthesis of Compound 50.

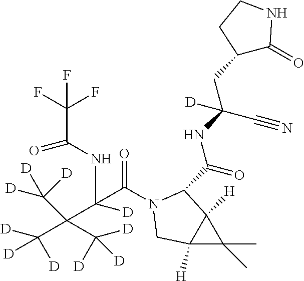 [SMILES: [2H]C([2H])([2H])C(C([2H])([2H])[2H])(C([2H])([2H])[2H])C([2H])(NC(=O)C(F)(F)F)C(=O)N1C[C@H]2[C@@H]([C@H]1C(=O)N[C@]([2H])(C#N)C[C@@H]1CCNC1=O)C2(C)C]

Chemical Formula: C₂₃H₂₁D₁₁F₃N₅O₄. Molecular Weight: 510.60. LC-MS (ESI, m/z, C₂₃H₂₁D₁₁F₃N₅O₄, 511, M+H).

¹H NMR (600 MHz, DMSO) δ: 9.44-9.41 (m, 1H), 9.05-9.01 (m, 1H), 7.68 (s, 1H), 4.15 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 62

### Synthesis of Compound 51.

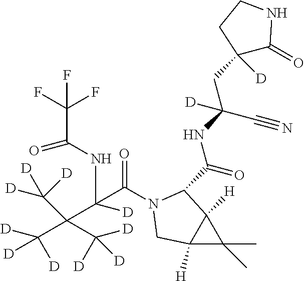 [SMILES: [2H]C([2H])([2H])C(C([2H])([2H])[2H])(C([2H])([2H])[2H])C([2H])(NC(=O)C(F)(F)F)C(=O)N1C[C@H]2[C@@H]([C@H]1C(=O)N[C@]([2H])(C#N)C[C@]1([2H])CCNC1=O)C2(C)C]

Chemical Formula: C₂₃H₂₀D₁₂F₃N₅O₄. Molecular Weight: 511.61. LC-MS (ESI, m/z, C₂₃H₂₀D₁₂F₃N₅O₄, 512, M+H).

¹H NMR (600 MHz, DMSO) δ: 9.45-9.40 (m, 1H), 9.05-9.00 (m, 1H), 7.68 (s, 1H), 4.15 (s, 1H), 3.92-3.90 (m, 1H), 3.70-3.68 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 63

### Synthesis of Compound 52.

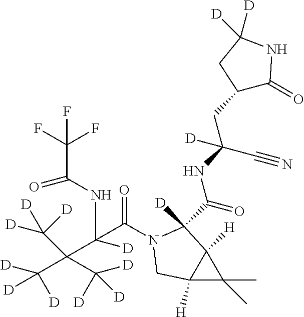 [SMILES: [2H]C1([2H])C[C@@H](C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1]

Chemical Formula: C₂₃H₁₈D₁₄F₃N₅O₄. Molecular Weight: 513.62. LC-MS (ESI, m/z, C₂₃H₁₈D₁₄F₃N₅O₄, 514, M+H).

¹H NMR (600 MHz, DMSO) δ: 9.45-9.41 (m, 1H), 9.05-9.02 (m, 1H), 7.68 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 64

### Synthesis of Compound 53.

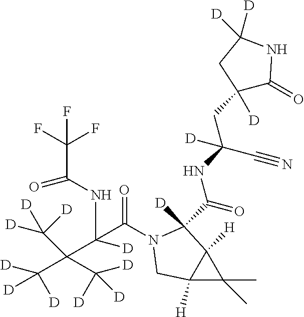 [SMILES: [2H]C1([2H])C[C@@]([2H])(C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1]

Chemical Formula: C₂₃H₁₇D₁₄F₅N₅O₄. Molecular Weight: 514.63. LC-MS (ESI, m/z, C₂₃H₁₇D₁₄F₅N₅O₄, 515, M+H).

¹H NMR (600 MHz, DMSO) δ: 9.45-9.40 (m, 1H), 9.05-9.01 (m, 1H), 7.68 (s, 1H), 3.92-3.90 (m, 1H), 3.70-3.68 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 65

### Synthesis of Compound 54.

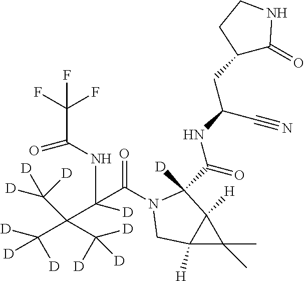 [SMILES: [2H]C([2H])([2H])C(C([2H])([2H])[2H])(C([2H])([2H])[2H])C([2H])(NC(=O)C(F)(F)F)C(=O)N1C[C@H]2[C@H](C2(C)C)[C@@]1([2H])C(=O)N[C@H](C#N)C[C@@H]1CCNC1=O]

Chemical Formula: C₂₃H₂₁D₁₁F₅N₅O₄. Molecular Weight: 510.60. LC-MS (ESI, m/z, C₂₃H₂₁D₁₁F₅N₅O₄, 511, M+H).

¹H NMR (600 MHz, DMSO) δ: 9.45-9.41 (m, 1H), 9.05-9.02 (m, 1H), 7.68 (s, 1H), 5.0-4.94 (m, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 66

### Synthesis of Compound 55.

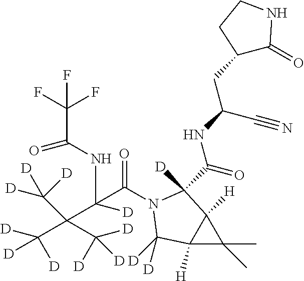 [SMILES: [2H]C1([2H])[C@H]2[C@H](C2(C)C)[C@@]([2H])(C(=O)N[C@H](C#N)C[C@@H]2CCNC2=O)N1C(=O)C([2H])(NC(=O)C(F)(F)F)C(C([2H])([2H])[2H])(C([2H])([2H])[2H])C([2H])([2H])[2H]]

Chemical Formula: C₂₃H₁₉D₁₃F₅N₅O₄. Molecular Weight: 512.61. LC-MS (ESI, m/z, C₂₃H₁₉D₁₃F₅N₅O₄, 513, M+H).

¹H NMR (600 MHz, DMSO) δ: 9.43-9.40 (m, 1H), 9.05-9.01 (m, 1H), 7.68 (s, 1H), 5.0-4.94 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 67

### Synthesis of Compound 56.

Chemical Formula: C₂₃H₁₉D₁₃F₅N₅O₄. Molecular Weight: 512.61. LC-MS (ESI, m/z,

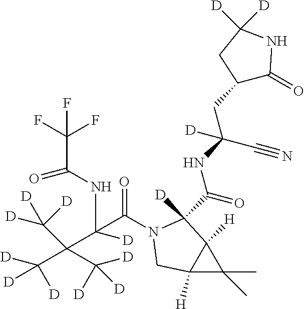 [SMILES: [2H]C1([2H])C[C@@H](C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1]

C₂₃H₁₉D₁₃F₅N₅O₄, 513, M+H).

¹H NMR (600 MHz, DMSO) δ: 9.45-9.40 (m, 1H), 9.06-9.02 (m, 1H), 7.68 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 68

### Synthesis of Compound 57.

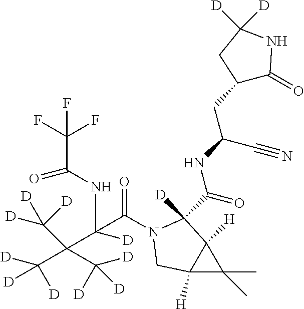 [SMILES: [2H]C1([2H])C[C@@H](C[C@@H](C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1]

Chemical Formula: C₂₃H₁₇D₁₅F₅N₅O. Molecular Weight: 514.63. LC-MS (ESI, m/z, C₂₃H₁₇D₁₅F₅N₅O₄, 515, M+H).

¹H NMR (600 MHz, DMSO) δ: 9.44-9.40 (m, 1H), 9.05-9.02 (m, 1H), 7.68 (s, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 69

### Synthesis of Compound 58.

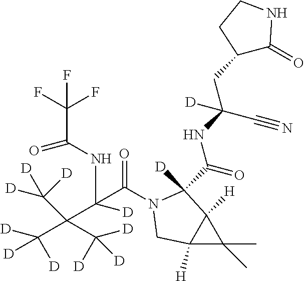 [SMILES: [2H]C([2H])([2H])C(C([2H])([2H])[2H])(C([2H])([2H])[2H])C([2H])(NC(=O)C(F)(F)F)C(=O)N1C[C@H]2[C@H](C2(C)C)[C@@]1([2H])C(=O)N[C@]([2H])(C#N)C[C@@H]1CCNC1=O]

Chemical Formula: C₂₃H₂₀D₁₂F₅N₅O. Molecular Weight: 511.61. LC-MS (ESI, m/z, C₂₃H₂₀D₁₂F₅N₅O₄, 512, M+H).

¹H NMR (600 MHz, DMSO) δ: 9.45-9.41 (m, 1H), 9.06-9.02 (m, 1H), 7.68 (s, 1H), 3.92-3.90 (m, 1H), 3.70-3.68 (m, 1H), 3.14 (m, 1H), 3.04 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 70

### Synthesis of Compound 59.

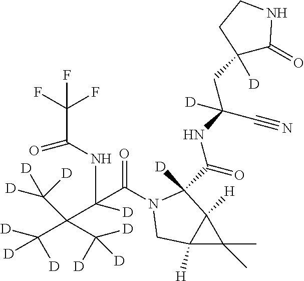 [SMILES: [2H]C([2H])([2H])C(C([2H])([2H])[2H])(C([2H])([2H])[2H])C([2H])(NC(=O)C(F)(F)F)C(=O)N1C[C@H]2[C@H](C2(C)C)[C@@]1([2H])C(=O)N[C@]([2H])(C#N)C[C@]1([2H])CCNC1=O]

Chemical Formula: C₂₃H₁₉D₁₃F₅N₅O₄. Molecular Weight: 512.61. LC-MS (ESI, m/z, C₂₃H₁₉D₁₃F₅N₅O₄, 513, M+H).

¹H NMR (600 MHz, DMSO) δ: 9.43-9.40 (m, 1H), 9.05-9.02 (m, 1H), 7.68 (s, 1H), 3.92-3.90 (m, 1H), 3.70-3.68 (m, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 71

### Synthesis of Compound 60.

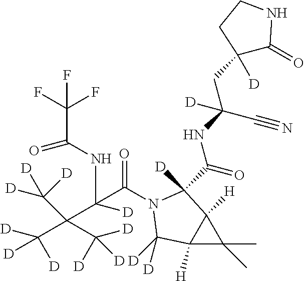 [SMILES: [2H]C1([2H])[C@H]2[C@H](C2(C)C)[C@@]([2H])(C(=O)N[C@]([2H])(C#N)C[C@]2([2H])CCNC2=O)N1C(=O)C([2H])(NC(=O)C(F)(F)F)C(C([2H])([2H])[2H])(C([2H])([2H])[2H])C([2H])([2H])[2H]]

Chemical Formula: C₂₃H₁₇D₁₅F₅N₅O₄. Molecular Weight: 514.63. LC-MS (ESI, m/z, C₂₃H₁₇D₁₅F₅N₅O₄, 515, M+H).

¹H NMR (600 MHz, DMSO) δ: 9.44-9.40 (m, 1H), 9.05-9.01 (m, 1H), 7.68 (s, 1H), 3.14 (m, 1H), 3.04 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 72

### Synthesis of Compound 61.

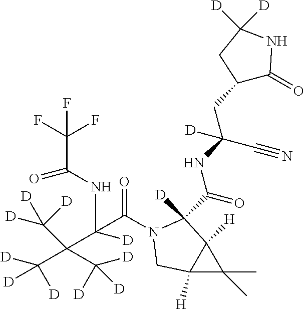 [SMILES: [2H]C1([2H])C[C@@H](C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1]

Chemical Formula: C₂₃H₁₈D₁₄F₅N₅O₄. Molecular Weight: 513.62. LC-MS (ESI, m/z, C₂₃H₁₈D₁₄F₅N₅O₄, 514, M+H).

¹H NMR (600 MHz, DMSO) δ: 9.45-9.41 (m, 1H), 9.05-9.02 (m, 1H), 7.68 (s, 1H), 3.92-3.90 (m, 1H), 3.70-3.68 (m, 1H), 2.42 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 73

### Synthesis of Compound 62.

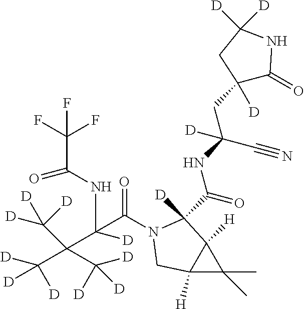 [SMILES: [2H]C1([2H])C[C@@]([2H])(C[C@@]([2H])(C#N)NC(=O)[C@]2([2H])[C@@H]3[C@H](CN2C(=O)C([2H])(NC(=O)C(F)(F)F)C(C([2H])([2H])[2H])(C([2H])([2H])[2H])C([2H])([2H])[2H])C3(C)C)C(=O)N1]

Chemical Formula: C₂₃H₁₇D₁₅F₅N₅O₄. Molecular Weight: 514.63. LC-MS (ESI, m/z, C₂₃H₁₇D₁₅F₅N₅O₄, 515, M+H).

¹H NMR (600 MHz, DMSO) δ: 9.44-9.40 (m, 1H), 9.05-9.02 (m, 1H), 7.68 (s, 1H), 3.92-3.90 (m, 1H), 3.70-3.68 (m, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 74

### Synthesis of Compound 63.

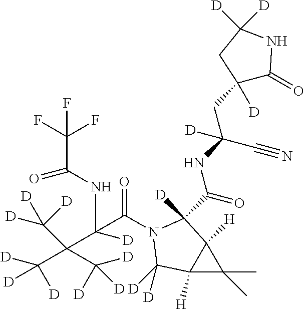 [SMILES: [2H]C1([2H])C[C@@]([2H])(C[C@@]([2H])(C#N)NC(=O)[C@]2([2H])[C@@H]3[C@@H](C3(C)C)C([2H])([2H])N2C(=O)C([2H])(NC(=O)C(F)(F)F)C(C([2H])([2H])[2H])(C([2H])([2H])[2H])C([2H])([2H])[2H])C(=O)N1]

Chemical Formula: C₂₃H₁₅D₁₇F₅N₅O₄. Molecular Weight: 516.64. LC-MS (ESI, m/z, C₂₃H₁₅D₁₇F₅N₅O₄, 517.3, M+H).

¹H NMR (600 MHz, DMSO) δ: 9.45-9.41 (m, 1H), 9.05-9.02 (m, 1H), 7.68 (s, 1H), 2.19 (m, 1H), 2.10 (m, 1H), 1.73 (s, 2H), 1.57 (m, 1H), 1.33-1.31 (m, 1H), 1.03 (s, 3H), 0.85 (s, 3H).

### Example 75

### In Vitro Anti-SARS-CoV-2 Virus Assay

Cell plating: Vero E6 cells were seeded in 12-well plates at 3×10⁵ cells/well, 10% FBS supplemented DMEM medium was added, and the plates were incubated overnight in a 37° C., 5% CO₂ incubator. Drug effect: After removing the cell culture media from the Vero cells in the 12-well plate, the VeroE6 cells were washed with PBS buffer and compound (final concentration 100 nM) was added to provide a 50 μL/well cell solution and the plate incubated in a 37° C., 5% CO₂ incubator for 1 hour. A 50 μL/well medium was set as a control. The cells were infected with the virus: After infecting the cells for 2 hours with SARS-CoV-2 virus, the virus and drug mixture were removed, and 10% FBS supplemented DMEM medium was added to the wells, and the cells were incubated in a 37° C., 5% CO₂ incubator for 2-3 days. PCR measurement: the supernatant of the culture medium was collected and retained in an incubator at 56° C. for 30 min. The viral RNA was extracted with a viral RNA extraction kit. The PCR reaction was performed with the viral nucleic acid detection kit following the instructions. The 2^{-ΔCT} value was calculated from the CT value displayed by the PCR instrument. The calculation formula of the virus replication inhibition rate was: (1−2^{-ΔCT})×100%, where the 2^{-ΔCT} value was the relative viral replication rate of the drug groups and the control group (tannic acid). The results are shown below in Table 2.

TABLE 2
The Inhibition of Virus Replication in the
Drug Groups and the Control Group

| | Com- | Com- | Com- | Com- | | |
|---|---|---|---|---|---|---|
| | pound | pound | pound | pound | PF- | |
| Group | 1 | 2 | 16 | 32 | 07321332 | Control |
| Inhibition | 71.5 | 73.5 | 72.6 | 70.4 | 72.9 | 54.1 |
| rate (%) | | | | | | |

### Example 76

### This Embodiment is the Detection of the Inhibition of the Protease Activity Targeting SARS-CoV-2 Virus M^{Pro}

Detection principle and mechanism of action: 3-chymotrypsin-like protease, the main protease (M^{Pro}, also known as 3CL^{Pro}), is encoded by ORF1 (localized to nsp5), located in the central region of the replicase gene. It is a key protein during the RNA replication of the novel coronavirus. When the new coronavirus invades the cell, the virus uses the host cell to synthesize two ultra-long replicase polypeptides (ppla and pplab) for self-replication. However, these replicase polypeptides need to be cleaved correctly into multiple proteins (e.g., RdRp, helicase, etc.), which are further assembled into the replication transcription machinery required for the virus to initiate replication of its own genetic material. These replicase polypeptides have at least 11 M^{Pro} cleavage sites, and only when such sites are correctly cleaved by M^{Pro}, the replication transcription machine can be assembled and viral replication can be initiated. Because of the important role of M^{Pro} protease during viral replication and the absence of any human protease counterparts for the virus to utilize, M^{Pro} has become a potential key drug target against the novel coronaviruses. The inhibitory activity on the SARS-CoV-2-M^{Pro} protease by the nucleoside derivatives was evaluated by a fluorescence resonance energy transfer method.

Specifically, the enzymatic reactions occurred in the wells of 96-well plates, and the total volume of the entire enzymatic reaction system was 120 μL, with the final concentration of protease at 30 nM and the final substrate concentration at 20 μM. The buffer solution of the reaction system included 50 mM Tris, 1 mM EDTA, pH 7.3. Substrate was added after the SARS-CoV-2-M^{Pro} protease and different concentrations of target compounds were incubated in the wells of 96-well plates at 30° C. for 10 min. After the substrate was added, the 96-well plates were placed immediately into a plate reader for measurement. Excitation and emission wavelengths were 340 nm and 405 nm, respectively, and fluorescence values were read every 30 seconds for 10 minutes. The final results were fitted to the reaction rate with a reading from the first 2 min and compared to the control group (DMSO) to calculate the inhibitory rates. IC₅₀ values of the SARS-CoV-2 viral nucleoside derivatives at corresponding time points were calculated using Graphpad prism 7 plot, which are shown below in Table 3.

TABLE 3
IC₅₀ Values for SARS-COV-2-M^{Pro} Protease

| Number | IC50 (μM) |
|---|---|
| Compound 1 | 2.948 ± 0.265 μM |
| Compound 2 | 2.786 ± 0.375 μM |
| Compound 16 | 2.723 ± 0.476 μM |
| Compound 32 | 2.835 ± 0.503 μM |
| PF-07321332 | 2.761 ± 0.382 μM |

### Example 77

### This Embodiment is a Pharmacokinetic Study of the Compounds in Rats.

Male Wistar-Hannover rats aged 7 to 10 weeks were used to conduct the pharmacokinetic study. All animals were housed individually during the pharmacokinetic study. Food and water were provided ad libitum (administration occurred when feeding). Animals were fasted overnight and fed 4 hours post-administration. Blood samples were collected via jugular vein cannula at predetermined time points. At study completion, animals were euthanized by overdose inhalation of anesthesia followed by exsanguination. Blood samples were collected into tubes containing K₂EDTA and stored on ice until plasma was obtained by centrifugation and stored in a freezer maintained at −20° C.

LC-MS/MS analysis of plasma samples: a protein precipitation method using 500/50 volume ratio of acetonitrile and methanol was developed to process the plasma samples. And reference substance were added into several blank plasma samples to afford various concentrations ranging from 0.1 ng/ml to 2500 ng/ml. Then a flat volume of internal standard propranolol (50 ng/ml) were added into each sample to be quantified. Internal standard method was performed to establish a calibration curve using the ratio of the peak intensity of reference substance to internal standard as ordinate and the concentration of reference substance as abscissa. Quantification was performed by analyzing plasma samples with LC-MS/MS, i.e. Waters ACQUITY ultra performance liquid chromatography system coupled with a Sciex 6500 triple quadrupole mass spectrometer. A Waters Acquity UPLC BEH C18 column (1.7 m, 2.150 mm) was used to separate the constituents. A gradient mobile phase was used to achieve good separation between analytes. Typically, the mobile phase consisted of solvent A (0.025% formic acid and 1 mM ammonium acetate in water/acetonitrile (95:5 v/v)) and solvent B (0.025% formic acid and 1 mM ammonium acetate in water/acetonitrile (5:95 v/v)) and the gradient generally started from 3˜30% B to approximately 1.2 minutes, increases to 50˜65% B to 1.6 minutes, and then decreases to 10-30% B until about 1.7˜1.9 minutes. Analyst 1.7 software was used for peak integration and standard curve regression.

Pharmacokinetic analysis: pharmacokinetic parameters were calculated using noncompartmental analysis (Watson v.7.5, Thermo Scientific). The area under the plasma concentration-time curve from t=0 to infinity (AUC_{0-∞}) was estimated using the linear trapezoidal rule. The results are shown below in Table 4. The data shows that the compounds of the invention have long half-lives and higher plasma exposures in animals and, thus, will have a better therapeutic effect than PF-07321332.

TABLE 4
pharmacokinetic parameters of the compound in rats

| | Dose | Cₘₐₓ | Tₘₐₓ | AUC_{0~∞} | T_{1/2} |
|---|---|---|---|---|---|
| Number | (mg/kg) | (ng/ml) | (h) | (ng · h/ml) | (h) |
| Compound 1 | 10 | 1393 | 2 | 1974 | 3.5 |
| Compound 2 | 10 | 1480 | 2 | 2325 | 4.2 |
| Compound 16 | 10 | 1452 | 2 | 2294 | 4 |
| Compound 32 | 10 | 1429 | 2 | 2011 | 3.5 |
| PF-07321332 | 10 | 1445 | 2 | 1982 | 2.9 |

### Example 78

### Preparation of Oral Tablets Containing Deuterated Cyano Compounds (Using Compound 2 as an Example).

The pharmaceutical carriers used for oral tablets are modifiers, fillers, binders, disintegrants, additives, glidants, lubricants, film coating materials, plasticizers, colorants, and the like.

| | | Content |
|---|---|---|
| Components | Effect | (mg/tablet) |
| Compound 2 | Pharmaceutical | 200 |
| | Ingredients | |
| Starch | Filler, Disintegrant | 100 |
| Calcium hydrogen | Filler | 20 |
| phosphate | | |
| Pregelatinized Starch | Filler | 40 |
| Citric acid | Modifier | 2 |
| Sodium bisulfite | Additives | 0.5 |
| 10% Starch Pulp | Binders | q.s. |
| Magnesium stearate | Lubricant | 1.5 |
| Opadry White | Coating premix | About 4 |
| Water, Ethanol | Solvent | q.s. |

### Operating Method:

Compound 2 was milled and sieved then mixed well with the filler, disintegrant, modifier, and additive. The 10% starch slurry was added to provide a soft material in a stirrer, the soft material was made into wet granules on a rocker, dried in an oven, mixed well with lubricant, and pressed into tablet cores. Film-coated tablets were obtained by coating the tablet cores with Opadry.

### Preparation of Capsules for Deuterated Cyano Compounds (Using Compound 2 as an Example).

The pharmaceutical carriers used for the capsules are fillers, binders, disintegrants, additives, lubricants, and the like.

| | | Content |
|---|---|---|
| Components | Effect | (mg/capsule) |
| Compound 2 | Pharmaceutical | 200 |
| | Ingredients | |
| Lactose Monohydrate | Filler | 82 |
| Pregelatinized Starch | Filler, Binder | 38 |
| Sodium carboxymethyl starch | Disintegrant | 12.5 |
| Magnesium stearate | Lubricant | 1.5 |

### Operating Method:

Compound 2 and each excipient were milled and sieved according to the above formula, and then mixed well with filler, binder in a certain proportion and disintegrant in partial proportion. The resulting mixture was then added to a dry granulator and compressed into strips, and the strips crushed into granules by a crusher. The granules were mixed well with an appropriate amount of lubricant and residual amount of disintegrant and filled into capsules.

Each of the technical features of the above described embodiments may be combined in any combination, and all possible combinations of each of the technical features in the above embodiments are not described in order to make the description concise, however, as long as there is no contradiction in the combination of these technical features, it should be considered within the scope of this specification.

The above described embodiments express only several embodiments of the invention, which are described in more specific and detail, but are understood as not limiting the scope of the invention. It should be noted that for those of ordinary skill in the art, a number of modifications and improvements may be made without departing from the inventive concept, all of which fall within the scope of protection of the invention.

What is claimed is:

1.-13. (canceled)

14. A deuterated cyano compound represented by Formula I: 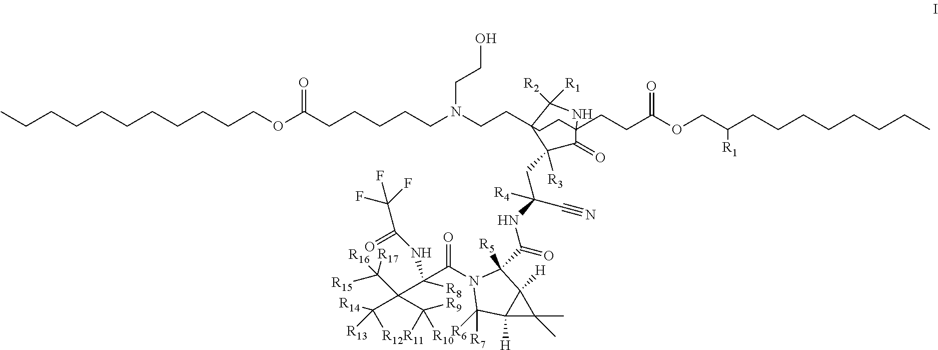
wherein R₁ to R₁₇ are each independently hydrogen or deuterium and at least one of R₁ to R₁₇ is deuterium;
or a pharmaceutically acceptable salt, isomer, or prodrug thereof.

15. The compound of claim 14, wherein at least one of R₁ to R₂ is deuterium.

16. The compound of claim 14, wherein at least one of R₄ to R₅ is deuterium.

17. The compound of claim 14, wherein at least one of R₆ to R₇ is deuterium.

18. The compound of claim 14, wherein R₈ is deuterium.

19. The compound of claim 14, wherein each of R₉ to R₁₇ are deuterium.

20. The compound of claim 14, selected from the group consisting of: 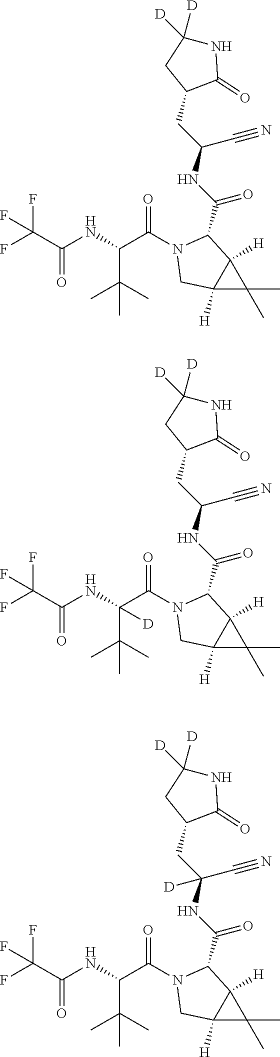 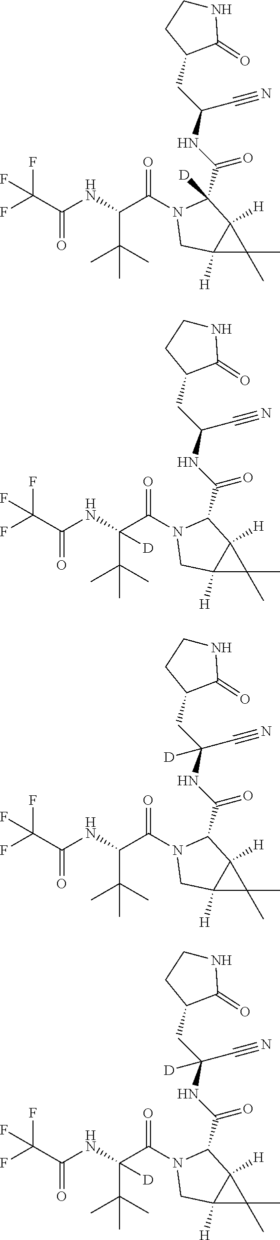 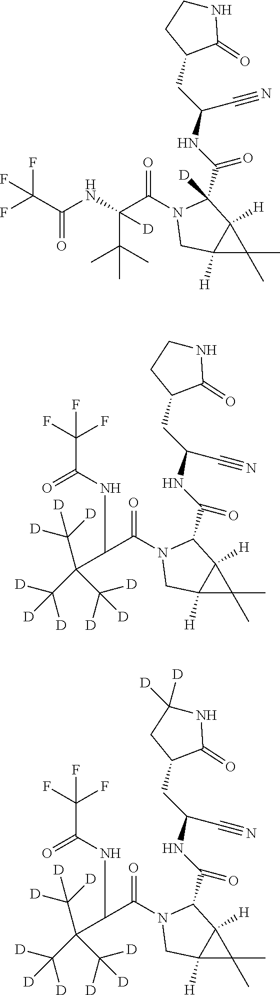 [SMILES: [2H][C@](NC(=O)C(F)(F)F)(C(=O)N1C[C@H]2[C@H](C2(C)C)[C@@]1([2H])C(=O)N[C@H](C#N)C[C@@H]1CCNC1=O)C(C)(C)C ; [2H]C([2H])([2H])C(C(NC(=O)C(F)(F)F)C(=O)N1C[C@H]2[C@@H]([C@H]1C(=O)N[C@H](C#N)C[C@@H]1CCNC1=O)C2(C)C)(C([2H])([2H])[2H])C([2H])([2H])[2H] ; [2H]C1([2H])C[C@@H](C[C@@H](C#N)NC(=O)[C@@H]2[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1] 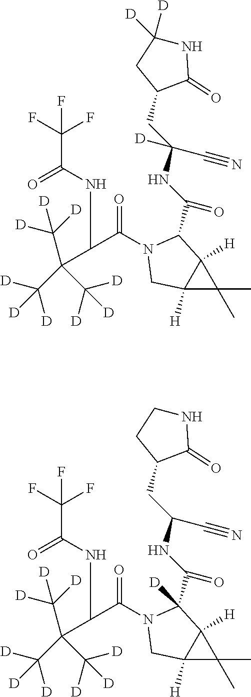 [SMILES: [2H]C1([2H])C[C@@H](C[C@@]([2H])(C#N)NC(=O)[C@@H]2[C@@H]3[C@H](CN2C(=O)C(NC(=O)C(F)(F)F)C(C([2H])([2H])[2H])(C([2H])([2H])[2H])C([2H])([2H])[2H])C3(C)C)C(=O)N1 ; [2H]C([2H])([2H])C(C(NC(=O)C(F)(F)F)C(=O)N1C[C@H]2[C@H](C2(C)C)[C@@]1([2H])C(=O)N[C@H](C#N)C[C@@H]1CCNC1=O)(C([2H])([2H])[2H])C([2H])([2H])[2H]]
or a pharmaceutically acceptable salt thereof.

21. A method for preparing the compound of formula I of claim 14, comprising the steps of: 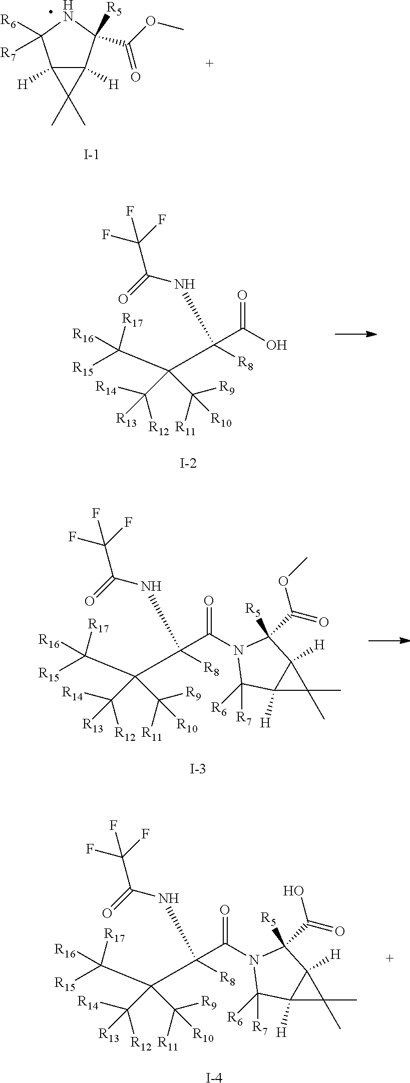 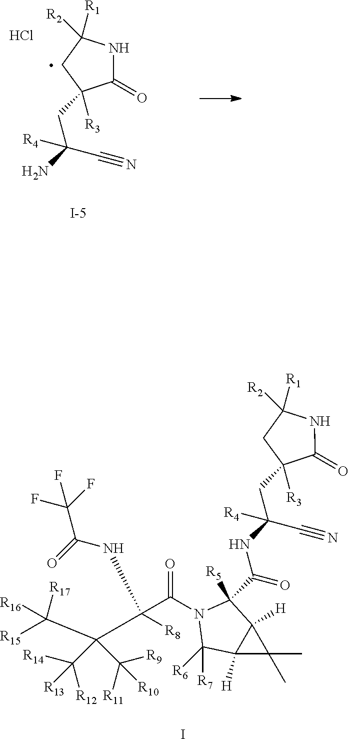
(1) condensing compound I-1 with compound I-2 in an organic solvent in the presence of a condensing agent to provide compound I-3;
(2) hydrolyzing compound I-3 in an organic solvent in the presence of alkali to provide compound I-4;
(3) condensing compound I-4 with compound I-5 in an organic solvent in the presence of a condensing agent to provide the compound of formula I.

22. A pharmaceutical composition comprising the compound as defined in claim 14, and a pharmaceutically acceptable carrier.

23. The pharmaceutical composition of claim 22, further comprising an antiviral drug.

24. A method of treating a viral infection in a human comprising administering to the human the pharmaceutical composition of claim 22.

25. The method of claim 24, wherein the viral infection is selected from the group consisting of human coronavirus, SARS-CoV-2, a SARS coronavirus, and a MERS coronavirus.

26. A method of treating a viral infection in a human comprising administering to the human the pharmaceutical composition of claim 23.

27. The method of claim 26, wherein the viral infection is selected from the group consisting of human coronavirus, SARS-CoV-2, a SARS coronavirus, and a MERS coronavirus.

28. A method of inhibiting 3CL protease comprising contacting the 3CL protease with a compound of claim 14.
